(19) United States
(12) Patent Application Publication
(10) Pub. No.: US 2014/0039031 A1
(43) Pub. Date: Feb. 6, 2014

(54) PHARMACEUTICAL FORMULATIONS OF ACETYL-11-KETO-B-BOSWELLIC ACID, DIINDOLYLMETHANE, AND CURCUMIN FOR PHARMACEUTICAL APPLICATIONS

(71) Applicants: Chris Brough, Austin, TX (US); Robert O. Williams, III, Austin, TX (US); James W. McGinity, Austin, TX (US); Dave A. Miller, Round Rock, TX (US); Justin Hughey, Hightstown, NJ (US); Ryan Bennett, Austin, TX (US)

(72) Inventors: Chris Brough, Austin, TX (US); Robert O. Williams, III, Austin, TX (US); James W. McGinity, Austin, TX (US); Dave A. Miller, Round Rock, TX (US); Justin Hughey, Hightstown, NJ (US); Ryan Bennett, Austin, TX (US)

(73) Assignees: Dispersol Technologies. LLC, Georgetown, TX (US); Board of Regents, The University of Texas System, Austin, TX (US)

(21) Appl. No.: 14/001,098

(22) PCT Filed: Feb. 23, 2012
(86) PCT No.: PCT/US12/26407

§ 371 Date: Oct. 18, 2013

Related U.S. Application Data
(60) Provisional application No. 61/445,950, filed on Feb. 23, 2011.
(60) Provisional application No. 61/551,361, filed on Oct. 25, 2011.

(51) Int. Cl. A61K 31/404; A61K 31/12; A61K 31/19

(52) U.S. Cl. CPC A61K 31/404; A61K 31/19; A61K 31/12
USPC 514/414; 514/557; 514/679

(57) ABSTRACT

The present disclosure is directed to compositions and methods for formulating a pharmaceutical dosage form by forming a composition comprising acetyl-11-keto-β-boswellic acid, diindolylmethane, or curcumin with one or more pharmaceutically acceptable excipients for enhanced solubility to increase bioavailability and improve therapeutic efficacy. The composition can be processed by thermo-kinetic compounding along with conventional methods known in the art, such as hot melt extrusion, melt granulation, compression molding, tablet compression, capsule filling, film-coating, or injection molding.

### PRIORITY CLAIM

This application claims priority to U.S. Provisional Application Ser. No. 61/445,950, filed on Feb. 23, 2011, and U.S. Provisional Application Ser. No. 61/551,361, filed on Oct. 25, 2011, the entire contents of each of which are incorporated herein by reference.

### RELATED APPLICATIONS

This application is related to U.S. Prov. Appl. Ser. No. 60/957,044, filed on Aug. 21, 2007, U.S. Prov. Appl. Ser. No. 61/050,922, filed on May 6, 2008, U.S. application Ser. No. 12/196,154, filed on Aug. 21, 2008, Int'l. Pat. Appl. PCT/US2008/073913, entitled “Thermo-Kinetic Mixing for Pharmaceutical Applications,” filed on Aug. 21, 2008, and U.S. Prov. Appl. Ser. No. 61/255,714, filed on Oct. 28, 2009.

### BACKGROUND OF THE INVENTION

1. Field of the Invention

The present disclosure relates in general to the field of pharmaceutical preparation and manufacturing, and more particularly, pharmaceutical formulations of acetyl-11-keto-β-boswellic acid, diindolylmethane, or curcumin to increase bioavailability and improve therapeutic efficacy, for example by preparing such formulating using thermokinetic compounding.

2. Description of Related Art

The beneficial applications of many potentially therapeutic molecules is often not fully realized either because they are abandoned during development due to poor pharmacokinetic profiles, or because of suboptimal product performance. Such problems may be due to poor solubility, which results in poor bioavailability. In recent years, the pharmaceutical industry has begun to rely more heavily on formulational methods for improving drug solubility. Consequently, advanced formulation technologies aimed at enhancing the dissolution properties of poorly water-soluble drugs are becoming increasingly important to modern drug delivery.

As set forth below, the following active pharmaceutical ingredients (“APIs”), acetyl-11-keto-β-boswellic acid (“AKBA”), diindolylmethane (“DIM”), and curcumin, are molecules with practical therapeutic applications.

AKBA is an organic acid extract of plants of the Boswellia genus, and has been used in traditional Indian ayurvedic medicine for the treatment of a number of inflammatory diseases, including osteoarthritis, chronic colitis, ulcerative colitis, Crohn's disease, and bronchial asthma. Studies suggest that AKBA has great potential for the treatment of inflammatory diseases and central nervous systems malignancies, if sufficient systemic concentrations can be achieved. It is also understood that AKBA possesses potent anti-inflammatory properties by inhibiting 5-lipoxygenase, human leukocyte elastase and the nuclear factor κB pathway, without exerting the adverse effects known for steroids. For example, AKBA has been indicated in the treatment of peritumoral brain edema accompanying brain tumors. AKBA has been found to induce apoptosis in prostate cancer cells, as well as to potently inhibit prostate tumor growth through inhibition of angiogenesis. AKBA has also shown effectiveness against multiple myeloma, colon cancer, prostate cancer, meningioma, ileitis, nociception and atherosclerosis. AKBA, however, shows poor bioavailability, which has been attributed mainly to its poor absorption.

DIM is derived from plants of the Brassica genus. Studies suggest that DIM has great potential for the treatment of numerous types of cancer and other diseases. For example, DIM has shown effectiveness against pancreatic cancer, breast cancer, arthritis and osteoclastogenesis, colon cancer, angiogenesis, prostate cancer, lung cancer, and human papilloma virus. Unfortunately, DIM shows poor bioavailability, which has been attributed to DIM's low solubility.

Curcumin is an extract of tumeric, a member of the ginger family, and has been used in traditional Indian ayurvedic medicine for the treatment of numerous diseases. Studies suggest that curcumin has antioxidant, anti-inflammatory, antiviral, antibacterial, antifungal, and anticancer properties that are mediated through the regulation of various transcription factors, growth factors, inflammatory cytokines, protein kinases, and other enzymes. Curcumin has shown effectiveness against pancreatic cancer, breast cancer, prostate cancer, colon cancer, Alzheimer's, depression, and hepatic disorders. Curcumin, however, shows poor bioavailability, which has been attributed to its low solubility and molecular instability.

Thus, while AKBA, DIM, and curcumin have numerous potential therapeutic applications, each of these APIs has poor solubility and bioavailability. Thus, currently available products with these APIs lack optimal performance. As a result, there is a great need in the medical and pharmaceutical industries to increase the solubility and bioavailability of these APIs to improve their therapeutic efficacy.

### SUMMARY OF THE INVENTION

The present disclosure is directed to pharmaceutical compositions which increase the bioavailability of AKBA, DIM, curcumin, or derivatives or analogs thereof, preferably, for example, by about one-and-a-half to at least ten times or more over currently available formulations. As a result, such formulations offer improved therapeutic efficacy. As disclosed herein, the pharmaceutical formulations may be oral or non-oral formulations.

Such formulations can be generated by using, for example, thermokinetic compounding (“TKC”). TKC offers numerous advantages for formulating APIs into pharmaceutical compositions such as brief processing times, low processing temperatures, high shear rates, and the ability to compound thermally incompatible materials into more homogenous composites. With these unique attributes, TKC offers a more efficient method of producing pharmaceutical compositions than traditional pharmaceutical processing operations, and in some instances permits the production of compositions that cannot be achieved by conventional methods. Thus, the application of TKC to pharmaceutical manufacturing of formulations containing AKBA, DIM, curcumin, any derivatives or analogs thereof, or any combination thereof, represents a substantial advance in terms of processing efficiency, compositional capabilities and properties, as well as commercial viability of dosage forms of advanced formulation design, e.g. solid dispersions. More particularly, with its advanced compounding ability, TKC permits compounding of AKBA, DIM, curcumin or derivatives or analogs thereof with excipients, adjuvants, or any combination thereof, to significantly increase bioavailability and improve therapeutic efficacy of the APIs.

The formulations of the present disclosure are useful in optimizing the medical, physiological, and therapeutic effects of AKBA, DIM, curcumin or derivatives or analogs thereof, for example including, but not limited to, the anti-inflammatory, anti-arthritis, anti-cancer, anti-oxidant, antiviral, antibacterial, antifungal, and anti-bronchial asthma activities of the APIs. AKBA, DIM, curcumin or derivatives or analogs thereof can also be used in the prevention and treatment of colitis, ileitis, Crohn's disease, depression, hepatic disorders, atherosclerotic cardiovascular disease and stroke.

In certain embodiments, the pharmaceutical compositions disclosed herein comprising AKBA may be used to prevent or treat diseases involving inflammation, cancer (e.g., human colorectal cancer), asthma, rheumatoid arthritis, ulcerative colitis, and other inflammatory diseases. In other embodiments, the pharmaceutical compositions disclosed herein comprising DIM may be used to prevent or treat cancer, respiratory papillomatosis, cervical dysplasia, as well as an immunostimulant against HPV. In still other embodiments, the pharmaceutical compositions disclosed herein comprising curcumin may be used to prevent or treat cancer (e.g., human colorectal cancer) and rheumatoid arthritis, as well as an anti-inflammatory agent.

In one aspect, the TKC process may increase the bioavailability of pharmaceutical formulations containing AKBA, DIM, curcumin or derivatives or analogs thereof by about one-and-a-half time, about two times, about three times, about four times, about five times, about six times, about seven times, about eight times, about nine times, or about ten times or more over currently available formulations. Such formulations therefore can improve the therapeutic efficacy of these APIs.

An embodiment of the present disclosure is directed to a method of making a pharmaceutical composition that includes AKBA, DIM, curcumin or derivatives or analogs thereof as an API and one or more pharmaceutically acceptable excipients or adjuvants by TKC, by thermokinetically processing the API with the one or more pharmaceutically acceptable excipients, adjuvants, or any combination thereof, into a composite. The pharmaceutical compositions disclosed herein can also include one or more APIs, for example a second API that complements the therapeutic benefits of AKBA, DIM, curcumin or derivatives or analogs thereof. In addition, the novel pharmaceutical composition or composite made by TKC may be further processed according to methods well known to those of skill in the art, including but not limited to hot melt extrusion, melt granulation, compression molding, tablet compression, capsule filling, film-coating, or injection molding into a final product. In certain embodiments, the composite made by TKC is the final product. Another embodiment is directed to addition of the API and one or more pharmaceutically acceptable excipients in a ratio of about 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, or 1:10. Yet another embodiment is directed to addition of the API and one or more pharmaceutically acceptable adjuvants in a ratio of about 1:2, 1:2.5, 1:3, 1:3.5, 1:4, 1:4.5, 1:5, 1:5.5, 1:6, 1:6.5, 1:7, 1:7.5, 1:8, 1:8.5, 1:9, 1:9.5, 1:10, 1:15, 1:20 1:25, 1:30, 1:40, 1:50, 1:60, 1:70, 1:80, 1:90, 1:100, 1:150, 1:200, 1:300, 1:400 or 1:500.

The thermokinetic processing may be conducted in a thermokinetic chamber. A thermokinetic chamber is an enclosed vessel or chamber in which TKC occurs. In one aspect, the average temperature inside the chamber is ramped up to a pre-defined final temperature over the duration of processing to achieve optimal thermokinetic mixing of the API and the one or more pharmaceutically acceptable excipients, adjuvants, or any combination thereof, into a composite. In another aspect, multiple speeds are used during a single, rotationally continuous TKC operation to achieve optimal thermokinetic mixing of the API and one or more pharmaceutically acceptable excipients, adjuvants, or any combination thereof, into a composite with minimal thermal degradation. The length of processing and exposure to elevated temperatures or speeds during thermokinetic mixing will generally be below the thermal sensitivity threshold of the API, excipient(s), or adjuvant(s). In another aspect, the thermokinetic processing is performed at an average temperature at or below the melting point of the API, excipient(s), or adjuvant(s); the thermokinetic processing is performed at an average temperature at or below the glass transition temperature of the API, excipient(s), or adjuvant(s); or the thermokinetic processing is performed at an average temperature at or below the molten transition point of the API, excipient(s), or adjuvant(s).

In one aspect, the composite made by TKC is a homogenous, heterogenous, or heterogeneously homogenous composite or an amorphous composite. In another aspect, the method, compositions and composite of the present disclosure may be adapted for oral or non-oral administration, for example buccal, sublingual, intravenous, parenteral, pulmonary, rectal, vaginal, topical, urethral, otic, ocular, or transdermal administration. In another aspect, the TKC may be conducted with or without a processing agent. Examples of processing agents include a plasticizer, a thermal lubricant, an organic solvent, an agent that facilitates melt blending, and an agent that facilitates downstream processing (e.g., lecithin). The composite may also include a carrier, e.g., a polymer with a high melt viscosity. In another aspect, the release rate profile of the API is determined by the one or more excipients of the composition. As such, the composition may be formulated for immediate release, mixed release, extended release or combinations thereof. In another aspect, the particle size of the API is reduced in an excipient/carrier system in which the API is not miscible, not compatible, or not miscible or compatible. In one aspect, the API is a nanocomposite with an excipient, a carrier, an adjuvant, or any combination thereof.

In certain embodiments, the thermokinetic processing substantially eliminates API, excipient or adjuvant degradation. For example, TKC may generate compositions and composites with less than about 2.0%, 1.0%, 0.75%, 0.5%, 0.1%, 0.05%, or 0.01% degradation products of each API or adjuvant. This advantage is important for AKBA, DIM and curcumin because they are thermally labile APIs, which typically undergo significant degradation during thermal processing, and are also subject to oxidation. In other embodiments, TKC may generate compositions with a minimum of at least about 90%, 91%, 92%, 93%, 94%, 95%, 96%, 97%, 98%, 99%, or 99.9% drug potency with respect to each API. Examples of TKC may be performed for less than 5, 10, 15, 20, 25, 30, 35, 40, 45, 50, 55, 60, 75, 100, 120, 150, 180, 240 and 300 seconds. Generally, TKC may be performed for less than 5, 10, 15, 20, 25, 30, 35, 40, 45, 50, 55, 60, 75, 100, 120, 150, 180, 240 and 300 seconds, and any ranges therein. In certain embodiments, the API has amorphous, crystalline, or intermediate morphology.

In certain embodiments, the formulations may provide for enhanced solubility of a poorly soluble API through the mixing of the API with pharmaceutically acceptable polymers, carriers, surfactants, excipients, adjuvants or any combination thereof. Thus, for example, compositions which display enhanced solubility are comprised of one or more APIs and a surfactant or surfactants, one or more APIs and a pharmaceutical carrier (thermal binder) or carriers, or one or more APIs and a combination of a surfactant and pharmaceutical carrier or surfactants and carriers. The API of this example may be AKBA, DIM, curcumin, derivatives or analogs thereof, or any combination thereof.

A further embodiment of the present disclosure is a pharmaceutical composition comprising AKBA, DIM, curcumin or derivatives or analogs thereof, and one or more pharmaceutically acceptable excipients, adjuvants, or a combination thereof, wherein a peak solubility of the API in the composition is greater than about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 20 μg/mL, about 25 μg/mL, about 30 μg/mL, about 35 μg/mL, about 40 μg/mL, 45 μg/mL, about 50 μg/mL or about 60 μg/mL in an aqueous buffer of pH between 4 and 8.

A further embodiment of the present disclosure is a pharmaceutical composition comprising AKBA, DIM, curcumin or derivatives or analogs thereof, and one or more pharmaceutically acceptable excipients, adjuvants, or a combination thereof, wherein a ratio of peak solubility of the API in the composition over peak solubility of the reference standard API is greater than about 3:1, about 4:1, about 5:1, about 6:1, about 7:1, about 8:1, about 9:1, or about 10:1.

A further embodiment of the present disclosure is a pharmaceutical composition comprising AKBA, DIM, curcumin or derivatives or analogs thereof, and one or more pharmaceutically acceptable excipients, wherein Cmax of the API in the composition and Cmax of the reference standard API when delivered orally have a ratio that is greater than about 5:1, about 6:1, about 7:1, about 8:1, about 10:1, about 12:1, about 15:1 or about 20:1.

A further embodiment of the present disclosure is a method of formulating a pharmaceutical composition comprising AKBA, DIM, curcumin or derivatives or analogs thereof, and one or more pharmaceutically acceptable excipients, adjuvants, or any combination thereof, by TKC to increase bioavailability of the API, comprising thermokinetic processing of the API with the one or more pharmaceutically acceptable excipients, adjuvants, or any combination thereof until melt blended into a composite.

A further embodiment of the present disclosure is a pharmaceutical composition comprising AKBA, DIM, curcumin or derivatives or analogs thereof, and one or more pharmaceutically acceptable excipients, adjuvants, or any combination thereof, wherein the composition is a homogenous, heterogenous, or heterogeneously homogenous composition in which the glass transition temperature is higher than the glass transition temperature of an identical combination of an identical API and pharmaceutically acceptable excipients, adjuvants, or any combination thereof processed thermally.

A further embodiment of the present disclosure is a pharmaceutical composition comprising AKBA, DIM, curcumin or derivatives or analogs thereof, and one or more pharmaceutically acceptable excipients, adjuvants, or any combination thereof, wherein the composition is a homogenous, heterogenous, or heterogeneously homogenous composition which has a single glass transition temperature, wherein an identical combination of an identical API and pharmaceutically acceptable excipients, adjuvants, or any combination thereof processed thermally has two or more glass transition temperatures.

A further embodiment of the present disclosure is a pharmaceutical composition comprising AKBA, DIM, curcumin or derivatives or analogs thereof, and one or more pharmaceutically acceptable excipients, adjuvants, or any combination thereof, processed into a composite, wherein the API is thermally labile, wherein the composite is a homogenous, heterogenous, or heterogeneously homogenous composition which has a less than about 1.0%, about 2%, about 3%, about 4% or about 5%, about 6%, about 7%, about 8%, about 9%, or about 10% degradation products of the thermally labile API.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. UV-Visible results of measuring the solubility enhancement of acetyl-11-keto-β-boswellic acid in pH 6.8 phosphate buffer by the composition having surfactants.

FIG. 2. UV-Visible results of measuring the solubility enhancement of acetyl-11-keto-β-boswellic acid in pH 6.8 phosphate buffer by the composition having polymer carriers (thermal binders).

FIG. 3. UV-Visible results of measuring the solubility enhancement of acetyl-11-keto-β-boswellic acid in pH 6.8 phosphate buffer by the composition having a combination of surfactants and polymer carriers (thermal binders).

FIG. 4. Processing data from the production of the AKBA formulations using TKC.

FIG. 5. Dissolution testing of formulations of AKBA produced using roto-evaporation under non-sink conditions.

FIG. 6. Dissolution testing of formulations of AKBA produced using thermo-kinetic compounding under non-sink conditions.

FIG. 6A. Comparison of dissolution testing of a formulation of AKBA produced using thermo-kinetic compounding with a currently marketed AKBA product under non-sink conditions.

FIG. 7. X-ray diffraction patterns of formulations of AKBA produced using roto-evaporation, PE is AKBA in the formulations, and the Plant Extract is crystalline AKBA.

FIG. 8. X-ray diffraction patterns of formulations of AKBA produced using thermo-kinetic compounding. DX is AKBA in the formulations and the Plant Extract is crystalline AKBA.

FIG. 9. Results of bioavailability of AKBA formulations from in vivo testing.

### DETAILED DESCRIPTION OF THE INVENTION

Although making and using various embodiments of the present disclosure are discussed in detail below, it should be appreciated that the present disclosure provides many inventive concepts that may be embodied in a wide variety of contexts. The specific aspects and embodiments discussed herein are merely illustrative of ways to make and use the disclosure, and do not limit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. With regard to the values or ranges recited herein, the term “about” is intended to capture variations above and below the stated number that may achieve substantially the same results as the stated number. In the present disclosure, each of the variously stated ranges is intended to be continuous so as to include each numerical parameter between the stated minimum and maximum value of each range. For example, a range of about 1 to about 4 includes about 1, 1, about 2, 2, about 3, 3, about 4, and 4. The terminology herein is used to describe specific embodiments of the disclosure, but their usage does not delimit the disclosure, except as outlined in the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “thermokinetic compounding” or “TKC” refers to a method of thermokinetic mixing until melt blended. TKC may also be described as a thermokinetic mixing process or thermokinetic processing in which processing ends at a point sometime prior to agglomeration.

As used herein, the phrase “a homogenous, heterogenous, or heterogeneously homogenous composite or an amorphous composite” refers to the various compositions that can be made using the TKC method.

As used herein, the term “heterogeneously homogenous composite” refers to a material composition having at least two different materials that are evenly and uniformly distributed throughout the volume.

As used herein, the phrase “reference standard active pharmaceutical ingredient” means the most thermodynamically stable form of the active pharmaceutical ingredient that is currently available.

Whether the composition of the present disclosure is a homogenous, heterogenous, or heterogeneously homogenous composition, an amorphous composition or combinations thereof, the TKC processing conditions can produce a composition with a glass transition temperature that is higher than the glass transition temperature of an identical combination of the identical API and pharmaceutically acceptable excipients, adjuvants, or any combination thereof, thermally processed, for example either with or without the use of a plasticizer. The TKC processing conditions can also produce a composition with a single glass transition temperature, wherein an identical combination of the identical API and pharmaceutically acceptable excipients, adjuvants, or any combination thereof, processed thermally, has two or more glass transition temperatures. In other embodiments, the pharmaceutical compositions of the present disclosure have a single glass transition temperature that is at least about 20%, 30%, 40%, 50%, 60%, 70%, 80%, or 90% higher than the lowest glass transition temperature of the identical combination processed thermally. Alternatively, the compositions made using thermokinetic processing may generate compositions with a minimum of at least about 90%, 91%, 92%, 93%, 94%, 95%, 96%, 97%, 98%, 99%, or 99.9% therapeutic potency with respect to each API.

As used herein, the term “thermokinetic chamber” refers to an enclosed vessel or chamber in which the TKC method is used to make the novel compositions of the present disclosure. In a TKC chamber the average temperature inside the chamber is ramped up to a pre-defined final temperature over the duration of processing to achieve thermokinetic compounding of the API and the one or more pharmaceutically acceptable excipients into a composite. The length of processing and exposure to elevated temperatures during thermokinetic compounding will generally be below the thermal sensitivity threshold of the API, the excipients or both. Multiple speeds may be used during a single, rotationally continuous TKC operation to achieve optimal thermokinetic mixing of the API and the one or more pharmaceutically acceptable excipients into a composite with minimal thermal degradation. The pre-defined final temperature and speed(s) are selected to reduce the possibility that the API, excipients and/or processing agents are degraded or their functionality is impaired during processing. Generally, the pre-defined final temperature, pressure, time of processing and other environmental conditions (e.g., pH, moisture, buffers, ionic strength, O₂) will be selected to substantially eliminate API, excipient, adjuvant and/or processing agent degradation.

In certain embodiments, the pharmaceutical formulations of the present disclosure are processed in a thermokinetic chamber as disclosed in U.S. Prov. Pat. Appl. Ser. No. 61/255,714, which is incorporated herein by reference. This disclosure is directed to a method of blending certain heat sensitive or thermolabile components in a thermokinetic mixer by using multiple speeds during a single, rotationally continuous operation on a batch containing thermolabile components in order to minimize any substantial thermal degradation, so that the resulting pharmaceutical compositions have increased bioavailability and stability. One embodiment is a method for continuous blending and melting of an autoheated mixture in the mixing chamber of a high speed mixer, where a first speed is changed mid-processing to a second speed upon achieving a first desired process parameter. Another embodiment is the use of variations in the shape, width and angle of the facial portions of the shaft extensions or projections that intrude into the main processing volume to control translation of rotational shaft energy delivered to the extensions or projections into heating energy within particles impacting the portions of the extensions or projections.

As used herein, “thermally processed” or “processed thermally” means that components are processed by hot melt extrusion, melt granulation, compression molding, tablet compression, capsule filling, film-coating, or injection molding.

As used herein, “extrusion” is the well-known method of applying pressure to a damp or melted composition until it flows through an orifice or a defined opening. The extrudable length varies with the physical characteristics of the material to be extruded, the method of extrusion, and the process of manipulation of the particles after extrusion. Various types of extrusion devices can be employed, such as screw, sieve and basket, roll, and ram extruders. Furthermore, the extrusion can be carried out through melt extrusion. Components of the present disclosure can be melted and extruded with a continuous, solvent free extrusion process, with or without inclusion of additives. Such processes are well-known to skilled practitioners in the art.

As used herein, “spray congealing” is a method that is generally used in changing the structure of materials, to obtain free flowing powders from liquids and to provide pellets. Spray congealing is a process in which a substance of interest is allowed to melt, disperse, or dissolve in a hot melt of other additives, and is then sprayed into an air chamber wherein the temperature is below the melting point of the formulation components, to provide congealed pellets. Such a process is well-known to skilled practitioners in the art.

As used herein, “solvent dehydration” or “spray drying technique” is commonly employed to produce a dry powder from a liquid or slurry by rapidly drying with a hot gas. This is one preferred method of drying many thermally-sensitive materials such as foods and pharmaceuticals. Water or organic solvent based formulations can be spray dried by using inert process gas, such as nitrogen, argon and the like. Such a process is well-known to skilled practitioners in the art.

In certain embodiments, the pharmaceutical formulations of the present disclosure can be processed by the techniques of extrusion, melt extrusion, spray congealing, spray drying or any other conventional technique to provide solid compositions from solution, emulsions suspensions or other mixtures of solids and liquids or liquids and liquids.

As used herein, “bioavailability” is a term meaning the degree to which a drug becomes available to the target tissue after being administered to the body. Poor bioavailability is a significant problem encountered in the development of pharmaceutical compositions, particularly those containing an API that is not highly soluble. In certain embodiments such as formulations of proteins, the proteins may be water soluble, poorly soluble, not highly soluble, or not soluble. The skilled artisan will recognize that various methodologies may be used to increase the solubility of proteins, e.g., use of different solvents, excipients, carriers, formation of fusion proteins, targeted manipulation of the amino acid sequence, glycosylation, lipidation, degradation, combination with one or more salts and the addition of various salts.

As used herein, the phrase “pharmaceutically acceptable” refers to molecular entities, compositions, materials, excipients, carriers, and the like that do not produce an allergic or similar untoward reaction when administered to humans in general.

As used herein, the term “active pharmaceutical ingredient” or “API” is interchangeable with the terms “drug,” “drug product,” “medication,” “liquid,” “biologic,” or “active ingredient.” As used herein, an “API” is any component intended to furnish pharmacological activity or other direct effect in the diagnosis, cure, mitigation, treatment, or prevention of disease, or to affect the structure or any function of the body of humans or other animals. In certain embodiments, the aqueous solubility of the API may be poorly soluble. As used herein, the term “composition” is interchangeable with the term “formulation.”

The API may be found in the form of one or more pharmaceutically acceptable salts, esters, derivatives, analogs, prodrugs, and solvates thereof. As used herein, a “pharmaceutically acceptable salt” is understood to mean a compound formed by the interaction of an acid and a base, the hydrogen atoms of the acid being replaced by the positive ion of the base.

A variety of administration routes are available for delivering the API to a patient in need. The particular route selected will depend upon the particular drug selected, the weight and age of the patient, and the dosage required for therapeutic effect. The pharmaceutical compositions may conveniently be presented in unit dosage form. The API suitable for use in accordance with the present disclosure, and its pharmaceutically acceptable salts, derivatives, analogs, prodrugs, and solvates thereof, can be administered alone, but will generally be administered in admixture with a suitable pharmaceutical excipient, adjuvant, diluent, or carrier selected with regard to the intended route of administration and standard pharmaceutical practice.

The API may be used in a variety of application modalities, including oral delivery as tablets, capsules or suspensions; pulmonary and nasal delivery; topical delivery as emulsions, ointments or creams; transdermal delivery; and parenteral delivery as suspensions, microemulsions or depot. As used herein, the term “parenteral” includes subcutaneous, intravenous, intramuscular, or infusion routes of administration.

The excipients and adjuvants that may be used in the presently disclosed compositions and composites, while potentially having some activity in their own right, for example, antioxidants, are generally defined for this application as compounds that enhance the efficiency and/or efficacy of the API. It is also possible to have more than one active ingredient in a given solution, so that the particles formed contain more than one active ingredient.

Any pharmaceutically acceptable excipient known to those of skill in the art may be used to produce the composites and compositions disclosed herein. Examples of excipients for use with the present invention include, but are not limited to, e.g., a pharmaceutically acceptable polymer, a thermolabile polymeric excipient, or a non-polymeric exicipient. Other non-limiting examples of excipients include, lactose, glucose, starch, calcium carbonate, kaoline, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used to enhance the efficacy and efficiency of the API. Additional non-limiting examples of compounds that can be included are binders, carriers, cryoprotectants, lyoprotectants, surfactants, fillers, stabilizers, polymers, protease inhibitors, antioxidants, bioavailability enhancers and absorption enhancers. The excipients may be chosen to modify the intended function of the active ingredient by improving flow, or bio-availability, or to control or delay the release of the API. Specific nonlimiting examples include: sucrose, trehaolose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor™ EL, Cremophor™ RH, Gelucire™ 50/13, Gelucire™ 53/10, Gelucire™ 44/14, Labrafil™, Solutol™ HS, dipalmitoyl phosphadityl choline, glycolic acid and salts, deoxycholic acid and salts, sodium fusidate, cyclodextrins, polyethylene glycols, Labrasol™, polyvinyl alcohols, polyvinyl pyrrolidones and tyloxapol. Using the process of the present disclosure, the morphology of the active ingredients can be modified, resulting in highly porous microparticles and nanoparticles.

Exemplary polymer carriers or thermal binders that may be used in the presently disclosed compositions and composites include but are not limited to polyethylene oxide; polypropylene oxide; polyvinylpyrrolidone; polyvinylpyrrolidone-co-vinylacetate; acrylate and methacrylate copolymers; polyethylene; polycaprolactone; polyethylene-co-polypropylene; alkylcelluloses such as methylcellulose; hydroxyalkylcelluloses such as hydroxymethylcellulose, hydroxyethylcellulose, hydroxypropylcellulose, and hydroxybutylcellulose; hydroxyalkyl alkylcelluloses such as hydroxyethyl methylcellulose and hydroxypropyl methylcellulose; starches, pectins; polysaccharides such as tragacanth, gum arabic, guar gum, and xanthan gum. One embodiment of the binder is poly(ethylene oxide) (PEO), which can be purchased commercially from companies such as the Dow Chemical Company, which markets PEO under the POLY OX™ exemplary grades of which can include WSR N80 having an average molecular weight of about 200,000; 1,000,000; and 2,000,000.

Suitable grades of PEO can also be characterized by viscosity of solutions containing fixed concentrations of PEO, such as for example:

| | |
|---|---|
| | Viscosity Range |
| | Aqueous Solution |
| POLYOX Water-Soluble Resin NF | at 25° C., mPa · s |

| | | |
|---|---|---|
| POLYOX Water-Soluble Resin NF | 30-50 | (5% solution) |
| WSR N-10 | | |
| POLYOX Water-Soluble Resin NF | 55-90 | (5% solution) |
| WSR N-80 | | |
| POLYOX Water-Soluble Resin NF | 600-1,200 | (5% solution) |
| WSR N-750 | | |
| POLYOX Water-Soluble Resin NF | 4,500-8,800 | (5% solution) |
| WSR-205 | | |
| POLYOX Water-Soluble Resin NF | 8,800-17,600 | (5% solution) |
| WSR-1105 | | |
| POLYOX Water-Soluble Resin NF | 400-800 | (2% solution) |
| WSR N-12K | | |
| POLYOX Water-Soluble Resin NF | 2,000-4,000 | (2% solution) |
| WSR N-60K | | |
| POLYOX Water-Soluble Resin NF | 1,650-5,500 | (1% solution) |
| WSR-301 | | |
| POLYOX Water-Soluble Resin NF | 5,500-7,500 | (1% solution) |
| WSR Coagulant | | |
| POLYOX Water-Soluble Resin NF | 7,500-10,000 | (1% solution) |
| WSR-303 | | |

Suitable polymer carriers or thermal binders that may or may not require a plasticizer include, for example, Eudragit™ RS PO, Eudragit™ S100, Kollidon™ SR (poly(vinyl acetate)-co-poly(vinylpyrrolidone) copolymer), Ethocel™ (ethylcellulose), HPC (hydroxypropylcellulose), cellulose acetate butyrate, poly(vinylpyrrolidone) (PVP), poly(ethylene glycol) (PEG), poly(ethylene oxide) (PEO), poly(vinyl alcohol) (PVA), hydroxypropyl methylcellulose (HPMC), ethylcellulose (EC), hydroxyethylcellulose (HEC), sodium carboxymethyl-cellulose (CMC), dimethylaminoethyl methacrylate-methacrylic acid ester copolymer, ethylacrylate-methylmethacrylate copolymer (GA-MMA), C-5 or 60 SH-50 (Shin-Etsu Chemical Corp.), cellulose acetate phthalate (CAP), cellulose acetate trimelletate (CAT), poly(vinyl acetate) phthalate (PVAP), hydroxypropylmethylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, Eudragit™ L-30-D (MA-EA, 1:1), Eudragit™ L100-55 (MA-EA, 1:1), Eudragit™ EPO (poly(butyl methacylate-co-(2-dimethylaminoethyl) methacrylate-co-methyl methacrylate) 1:2:1), hydroxypropylmethylcellulose acetate succinate (HPMCAS), Coateric™ (PVAP), Aquateric™ (CAP), and AQUACOAT™ (HPMCAS), Soluplus™ (polyvinyl caprolactam-polyvinyl acetate-polyethylene glycol graft copolymer, BASF), Luvitec™ K 30 (polyvinylpyrrolidone, PVP), Kollidon™ (polyvinylpyrrolidone, PVP), polycaprolactone, starches, pectins; polysaccharides such as tragacanth, gum arabic, guar gum, and xanthan gum.

The stabilizing and non-solubilizing carrier may also contain various functional excipients, such as: hydrophilic polymer, antioxidant, super-disintegrant, surfactant including amphiphilic molecules, wetting agent, stabilizing agent, retardant, similar functional excipient, or combination thereof, and plasticizers including citrate esters, polyethylene glycols, PG, triacetin, diethylphthalate, castor oil, and others known to those or ordinary skill in the art. Extruded material may also include an acidifying agent, adsorbent, alkalizing agent, buffering agent, colorant, flavorant, sweetening agent, diluent, opaquant, complexing agent, fragrance, preservative or a combination thereof.

Exemplary hydrophilic polymers which can be a primary or secondary polymeric carrier that can be included in the composites or composition disclosed herein include poly(vinyl alcohol) (PVA), polyethylene-polypropylene glycol (e.g. POLOXAMER™), carbomer, polycarbophil, or chitosan. Hydrophilic polymers for use with the present disclosure may also include one or more of hydroxypropyl methylcellulose, carboxymethylcellulose, hydroxypropyl cellulose, hydroxyethyl cellulose, methylcellulose, natural gums such as gum guar, gum acacia, gum tragacanth, or gum xanthan, and povidone. Hydrophilic polymers also include polyethylene oxide, sodium carboxymethycellulose, hydroxyethyl methyl cellulose, hydroxymethyl cellulose, carboxypolymethylene, polyethylene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), carrageenate alginates, carbomer, ammonium alginate, sodium alginate, or mixtures thereof.

Exemplary adjuvants that can be included in the composites or composition disclosed herein include non-specific inhibitors of metabolic enzymes. Examples of these enzyme inhibitors can include inhibitors of cytochrome P-450 enzymes (CYP) and inhibitors of monoamine oxidase enzymes. Monoamine oxidase inhibitors that may be used in the formulations of the present disclosure include one or more of iproniazid, isocarboxazid, phenelzine, tranylcypromine, befloxatone, brofaromine, moclobemide, piperine, pirlindole, toloxatone, or any combination thereof. Cytochrome P-450 inhibitors that may be used in the formulations of the present disclosure include one or more of the following, for example:

Cytochrome P450 (CYP) Inhibitors

| 1A2 | 2B6 | NAT2 | 2C19 |
|---|---|---|---|
| acyclovir | clopidogrel | acetaminophen | artemisinin |
| amiodarone | efavirenz | | chloramphenicol |
| caffeine | fluoxetine | | delavirdine |
| cimetidine | fluvoxamine | | efavirenz |
| ciprofloxacin | ketoconazole | | esomeprazole |
| enoxacin | memantine | | felbamate |
| echinacea | nelfinavir | | fluconazole |
| enoxacin | oral contracep- | | fluoxetine |
| famotidine | tives | | fluvoxamine |
| flutamide | paroxetine | | indomethacin |
| fluvoxamine | ritonavir | | inh |
| grapefruit juice | thiotepa | | modafinil |
| lidocaine | ticlopidine | | Provigil |
| lomefloxacin | | | omeprazole |
| mexiletine | | | oral contracep- |
| moclobemide | | | tives |
| norfloxacin | | | oxcarbazepine |
| ofloxacin | | | ticlopidine |
| oral contracep- | | | topiramate |
| tives | | | voriconazole |
| perphenazine | | | |
| phenacetin | | | |
| propafenone | | | |
| ropinirole | | | |
| tacrine | | | |
| ticlopidine | | | |
| tocainide | | | |
| verapamil | | | |
| zileuton | | | |

| 2C9 | 2D6 | 3A4, 5, 7 |
|---|---|---|
| amiodarone | amiodarone | amiodarone |
| anastrazole | amitriptyline} | amprenavir |
| cimetidine | bupropion | aprepitant -initially |
| delavirdine | celecoxib | atazanavir |
| efavirenz | chlorpheniramine | cimetidine} |
| fenofibrate | chlorpromazine | ciprofloxacin |
| fluconazole | cimetidine | clarithromycin |
| fluoxetine | cinacalcet | delavirdine |
| fluvoxamine | citalopram} | diltiazem |
| fluvastatin | chlorpheniramine | doxycycline |
| isoniazid | clomipramine | echinacea |
| ketoconazole | desipramine | enoxacin |
| leflunomide | diphenhydramine | erythromycin |
| modafiinil | doxepin | fluconazole |
| phenylbutazone | duloxetine | fluvoxamine |
| sertraline | fluvoxamine} | grapefruit extract |
| sulfamethoxazole | fluoxetine | grapefruit juice |
| sulfaphenazole | goldenseal | imatinib |
| tamoxifen | halofantrine | indinavir |
| teniposide | haloperidol | itraconazole |
| valproic acid | hydroxyzine | ketoconazole |
| voriconazole | imipramine | miconazole |
| zafirlukast, | methadone | nefazodone |
| 5-fluorouracil | metoclopramide | nelfinavir |
| | moclobemide | piperine |
| | paroxetine | ritonavir |
| | pimozide | saquinavir |
| | propafenone | telithromycin |
| | quinidine/quinine | verapamil |
| | ritonavir | voriconazole |
| | sertraline | |
| | terbinafine | |
| | thioridazine | |
| | ticlopidine | |

As used herein, “poorly soluble” refers to an API having a solubility such that the dose to be administered can be dissolved in 250 ml of aqueous media ranging in pH from 1 to 7.5, an API with a slow dissolution rate, and an API with a low equilibrium solubility, for example resulting in decreased bioavailability of the pharmacological effect of the therapeutic API being delivered.

As used herein, “derivative” refers to chemically modified inhibitors or stimulators that still retain the desired effect or property of the original API. Such derivatives may be derived by the addition, removal, or substitution of one or more chemical moieties on the parent molecule. Such moieties may include, but are not limited to, an element such as a hydrogen or a halide, or a molecular group such as a methyl group. Such a derivative may be prepared by any method known to those of skill in the art. The properties of such derivatives may be assayed for their desired properties by any means known to those of skill in the art. As used herein, “analogs” include structural equivalents or mimetics.

The solution agent used in the solution can be an aqueous such as water, one or more organic solvents, or a combination thereof. When used, the organic solvents can be water miscible or non-water miscible. Suitable organic solvents include but are not limited to ethanol, methanol, tetrahydrofuran, acetonitrile, acetone, tert-butyl alcohol, dimethyl sulfoxide, N,N-dimethyl formamide, diethyl ether, methylene chloride, ethyl acetate, isopropyl acetate, butyl acetate, propyl acetate, toluene, hexanes, heptane, pentane, and combinations thereof.

By “immediate release” is meant a release of an active agent to an environment over a period of seconds to no more than about 30 minutes once release has begun and release begins within no more than about 2 minutes after administration. An immediate release does not exhibit a significant delay in the release of drug.

By “rapid release” is meant a release of an active agent to an environment over a period of 1-59 minutes or 0.1 minute to three hours once release has begun and release can begin within a few minutes after administration or after expiration of a delay period (lag time) after administration.

As used herein, the term “extended release” profile assumes the definition as widely recognized in the art of pharmaceutical sciences. An extended release dosage form will release the API at a substantially constant rate over an extended period of time or a substantially constant amount of drug will be released incrementally over an extended period of time. An extended release tablet generally effects at least a two-fold reduction in dosing frequency as compared to the drug presented in a conventional dosage form (e.g., a solution or rapid releasing conventional solid dosage forms).

By “controlled release” is meant a release of an active agent to an environment over a period of about eight hours up to about 12 hours, 16 hours, 18 hours, 20 hours, a day, or more than a day. By “sustained release” is meant an extended release of an active agent to maintain a constant drug level in the blood or target tissue of a subject to which the device is administered.

The term “controlled release”, as regards to drug release, includes the terms “extended release”, “prolonged release”, “sustained release”, or “slow release”, as these terms are used in the pharmaceutical sciences. A controlled release can begin within a few minutes after administration or after expiration of a delay period (lag time) after administration.

A slow release dosage form is one that provides a slow rate of release of drug so that drug is released slowly and approximately continuously over a period of 3 hr, 6 hr, 12 hr, 18 hr, a day, 2 or more days, a week, or 2 or more weeks, for example.

The term “mixed release” as used herein refers to a pharmaceutical agent that includes two or more release profiles for one or more active pharmaceutical ingredients. For example, the mixed release may include an immediate release and an extended release portion, each of which may be the same API or each may be a different API.

A timed release dosage form is one that begins to release an API after a predetermined period of time as measured from the moment of initial exposure to the environment of use.

A targeted release dosage form generally refers to an oral dosage form that is designed to deliver an API to a particular portion of the gastrointestinal tract of a subject. An exemplary targeted dosage form is an enteric dosage form that delivers a drug into the middle to lower intestinal tract but not into the stomach or mouth of the subject. Other targeted dosage forms can deliver to other sections of the gastrointestinal tract such as the stomach, jejunum, ileum, duodenum, cecum, large intestine, small intestine, colon, or rectum.

By “delayed release” is meant that initial release of an API occurs after expiration of an approximate delay (or lag) period. For example, if release of an API from an extended release composition is delayed two hours, then release of the API begins at about two hours after administration of the composition, or dosage form, to a subject. In general, a delayed release is opposite of an immediate release, wherein release of an API begins after no more than a few minutes after administration. Accordingly, the API release profile from a particular composition can be a delayed-extended release or a delayed-rapid release. A “delayed-extended” release profile is one wherein extended release of an API begins after expiration of an initial delay period. A “delayed-rapid” release profile is one wherein rapid release of an API begins after expiration of an initial delay period.

A pulsatile release dosage form is one that provides pulses of high active ingredient concentration, interspersed with low concentration troughs. A pulsatile profile containing two peaks may be described as “bimodal.” A pulsatile profile of more than two peaks may be described as multi-modal.

A pseudo-first order release profile is one that approximates a first order release profile. A first order release profile characterizes the release profile of a dosage form that releases a constant percentage of an initial drug charge per unit time.

A pseudo-zero order release profile is one that approximates a zero-order release profile. A zero-order release profile characterizes the release profile of a dosage form that releases a constant amount of drug per unit time.

The resulting composites or compositions disclosed herein may also be formulated to exhibit enhanced dissolution rate of a formulated poorly water soluble drug.

Compositions of an API that enhance solubility may comprise a mixture of the API and an additive that enhances the solubility of the API. Examples of such additives include but are not limited to surfactants, polymer carriers, pharmaceutical carriers, thermal binders or other excipients. A particular example may be a mixture of an API with a surfactant or surfactants, an API with a polymer or polymers, or an API with a combination of a surfactant and polymer carrier or surfactants and polymer carriers. A further example is a composition where the API is AKBA, DIM, curcumin, or derivatives or analogs thereof.

Surfactants that can be used in the disclosed compositions to enhance solubility have been previously presented. Particular examples of such surfactants include but are not limited to sodium dodecyl sulfate, dioctyl docusate sodium, Tween 80, Span 20, Cremophor™ EL or Vitamin E TPGS. Polymer carriers that can be used in the disclosed composition to enhance solubility have been previously presented. Particular examples of such polymer carriers include but are not limited to Soluplus™, Eudragit™ L100-55, Eudragit™ EPO, Kollidon™ VA 64, Luvitec™ K 30, Kollidon™, AQOAT™-HF, and AQOAT™-LF. The composition of the present disclosure can thus be any combination of one or more of the APIs, zero, one or more of surfactants or zero, one or more of polymers presented herein.

Solubility can be indicated by peak solubility, which is the highest concentration reached of a species of interest, for example an API, over time during a solubility experiment conducted in a specified medium. The enhanced solubility can be represented as the ratio of peak solubility of the API in a pharmaceutical composition of the present disclosure compared to peak solubility of the reference standard API under the same conditions. Preferable, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of an API that enhance bioavailability may comprise a mixture of the API and one or more pharmaceutically acceptable adjuvants that enhance the bioavailability of the API. Examples of such adjuvants include but are not limited to enzymes inhibitors. Particular examples are such enzyme inhibitors include but are not limited to inhibitors that inhibit cytochrome P-450 enzyme and inhibitors that inhibit monoamine oxidase enzyme. Bioavailability can be indicated by the Cmax of the API as determined during in vivo testing, where Cmax is the highest reached blood level concentration of the material of interest, such as an API, over time of monitoring. Enhanced bioavailability can be represented as the ratio of Cmax of the API in a pharmaceutical composition of the present disclosure compared to Cmax of the reference standard API under the same conditions. This Cmax ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

An example of a composition or formulation having a stable release profile follows. Two tablets having the same formulation are made. The first tablet is stored for one day under a first set of conditions, and the second tablet is stored for four months under the same first set of conditions. The release profile of the first tablet is determined after the single day of storage and the release profile of the second tablet is determined after the four months of storage. If the release profile of the first tablet is approximately the same as the release profile of the second tablet, then the tablet/film formulation is considered to have a stable release profile.

Another example of a composition or formulation having a stable release profile follows. Tablets A and B, each comprising a composition according to the invention, are made, and Tablets C and D, each comprising a composition not according to the invention, are made. Tablets A and C are each stored for one day under a first set of conditions, and tablets B and D are each stored for three months under the same first set of conditions. The release profile for each of tablets A and C is determined after the single day of storage and designated release profiles A and C, respectively. The release profile for each of tablet B and D is determined after the three months of storage and designated release profiles B and D, respectively. The differences between release profiles A and B are quantified as are the differences between release profiles C and D. If the difference between the release profiles A and B is less than the difference between release profiles C and D, tablets A and B are understood to provide a stable or more stable release profile.

Specifically, the TKC process can be used for one or more of the following pharmaceutical applications.

Dispersion of an API, wherein the API is AKBA, DIM, curcumin, or derivatives or analogs thereof, in polymeric and/or non-polymeric pharmaceutically acceptable materials for the purpose of delivering the API to a patient via oral, pulmonary, parenteral, vaginal, rectal, urethral, transdermal, or topical routes of delivery.

Dispersion of an API, wherein the API is AKBA, DIM, curcumin, or derivatives or analogs thereof, in polymeric and/or non-polymeric pharmaceutically acceptable materials for the purpose of improving the oral delivery of the API by improving the bioavailability of the API, extending the release of the API, targeting the release of the API to specific sites, delaying the release of the API, or producing pulsatile release systems for the API.

Dispersion of an API, wherein the API is AKBA, DIM, curcumin, or derivatives or analogs thereof, in polymeric and/or non-polymeric pharmaceutically acceptable materials for the purpose of creating bioerodable, biodegradable, or controlled release implant delivery devices.

Producing solid dispersions of a thermolabile API, wherein the API is AKBA, DIM, curcumin, or derivatives or analogs thereof, by processing at low temperatures for very brief durations.

Producing solid dispersions of an API, wherein the API is AKBA, DIM, curcumin, or derivatives or analogs thereof, in thermolabile polymers and excipients by processing at low temperatures for very brief durations.

Rendering an API, wherein the API is AKBA, DIM, curcumin, or derivatives or analogs thereof, amorphous while dispersing in a polymeric, non-polymeric, or combination excipient carrier system.

Rendering an API, wherein the API is AKBA, DIM, curcumin, or derivatives or analogs thereof, amorphous while dispersing in a polymeric, non-polymeric, or combination excipient carrier system and adjuvants.

Dry milling of a crystalline API, wherein the API is AKBA, DIM, curcumin, or derivatives or analogs thereof, to reduce the particle size of the bulk material.

Wet milling of a crystalline API, wherein the API is AKBA, DIM, curcumin, or derivatives or analogs thereof, with a pharmaceutically acceptable solvent to reduce the particle size of the bulk material.

Melt milling of a crystalline API, wherein the API is AKBA, DIM, curcumin, or derivatives or analogs thereof, with one or more molten pharmaceutical excipients having limited miscibility with the crystalline API to reduce the particle size of the bulk material.

Milling a crystalline API, wherein the API is AKBA, DIM, curcumin, or derivatives or analogs thereof, in the presence of polymeric or non-polymeric excipient to create ordered mixtures where fine drug particles adhere to the surface of excipient particles and/or excipient particles adhere to the surface of fine drug particles.

Producing single phase, miscible composites of two or more pharmaceutical materials previously considered to be immiscible for utilization in a secondary processing step, e.g. melt extrusion, film coating, tableting and granulation.

Pre-plasticizing polymeric materials for subsequent use in film coating or melt extrusion operations.

Rendering a crystalline or semi-crystalline pharmaceutical polymer amorphous, which can be used as a carrier for an API, wherein the API is AKBA, DIM, curcumin, or derivatives or analogs thereof, in which the amorphous character improves the dissolution rate of the API-polymer composite, the stability of the API-polymer composite, and/or the miscibility of the API and the polymer.

Deaggregating and dispersing engineered particles in a polymeric carrier without altering the properties of the engineered particles.

Simple blending of an API, wherein the API is AKBA, DIM, curcumin, or derivatives or analogs thereof, in powder form with one or more pharmaceutical excipients.

Producing composites comprising a high melting point API, wherein the API is AKBA, DIM, curcumin, or derivatives or analogs thereof, and one or more thermolabile polymers without the use of processing agents.

Homogenously dispersing a coloring agent or opacifying agent within a polymer carrier or excipient blend.

Additionally, compositions of the present disclosure may be processed using any technique known to one skilled in the art to produce a solid formulation, including fusion or solvent based techniques. Specific examples of these techniques include extrusion, melt extrusion, hot-melt extrusion, spray congealing, spray drying, hot-spin mixing, ultrasonic compaction, and electrostatic spinning.

### EXAMPLES

It will be understood that particular embodiments described herein are shown by way of illustration and not as limitations of the invention. The principal features of this invention can be employed in various embodiments without departing from the scope of the invention. All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### Example 1

Evaluation of Solubility Enhancement Capability of Surfactants for Solubilizing AKBA.

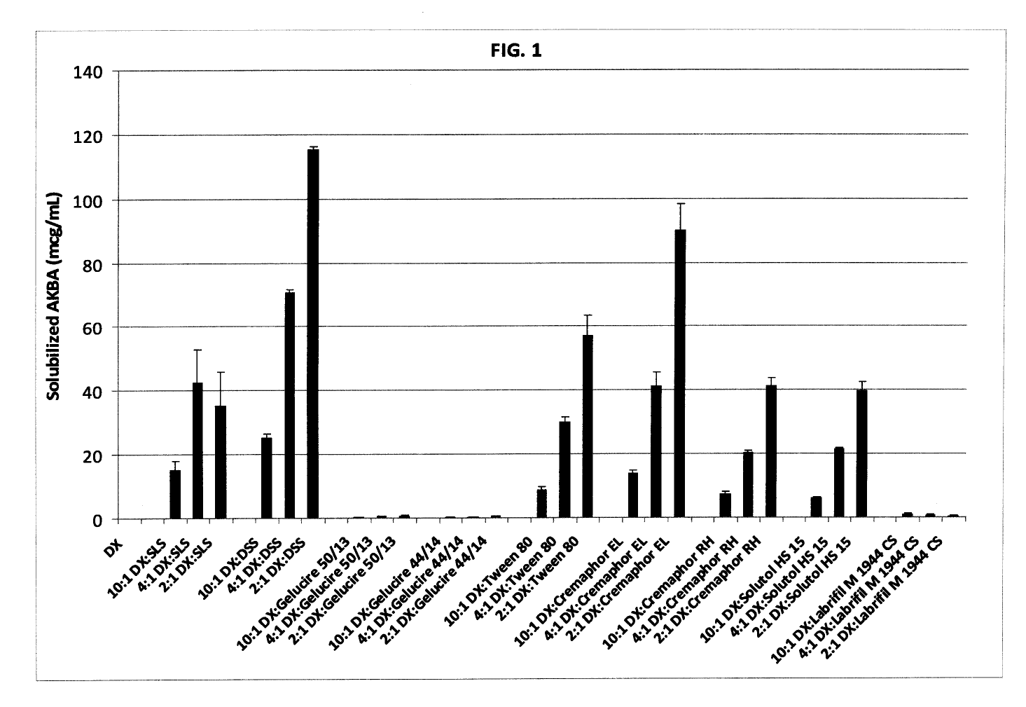

100 mg of AKBA was placed in a 10 mL cuvette, followed by the desired amount of surfactant to be studied to give the desired ratio of materials. Powder surfactants were measured directly into the cuvette in the amounts of 10 mg, 25 mg and 50 mg. Liquid surfactants were added by making a solution of 1.25 gm of surfactant in 250 ml of buffer, and then adding 2 ml, 5 ml, or 10 ml of the buffer solution to add 10 mg, 25 mg and 50 mg of the surfactant. A pH 6.8 phosphate buffer was added as needed to bring the volume up to 10 mL. After all material was added to the cuvette, the cuvette was sealed, sonicated for 2 hours on two separate occasions and then shaken for at least 72 hours at 37° C. The resulting material was filtered through a 0.20 micron filter and analyzed by UV-Visible spectroscopy to determine the AKBA content. Results are given in Table 1 and FIG. 1.

### Example 2

Evaluation of Solubility Enhancement Capability of Polymer Carriers (Thermal Binders) for Solubilizing AKBA.

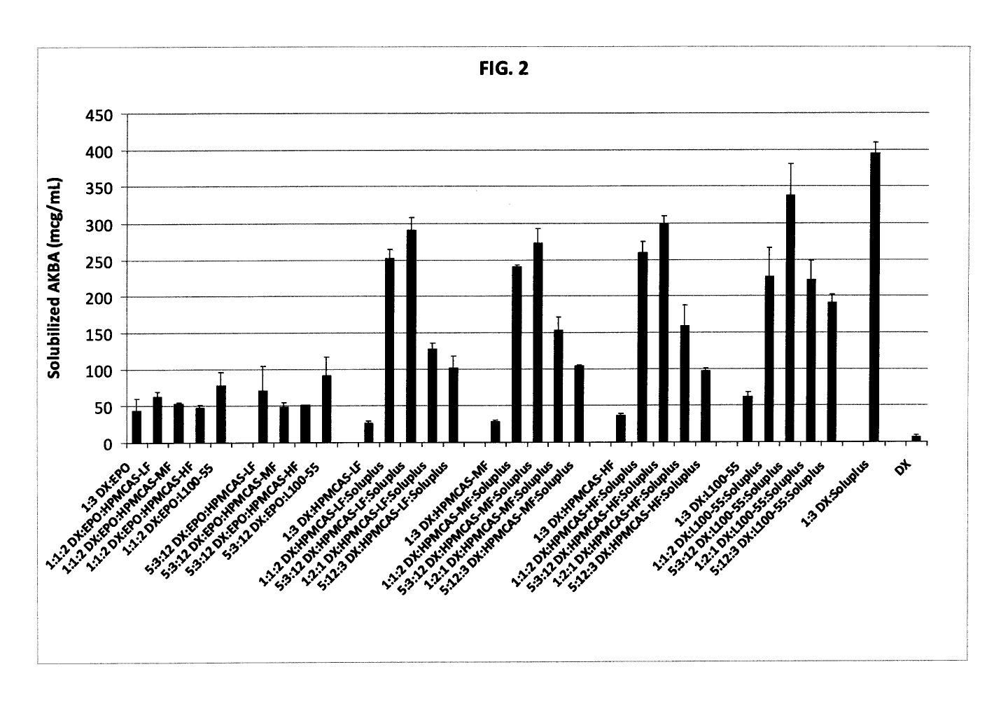

20 mg of AKBA was placed in a 10 mL cuvette, followed by the desired amount of polymer carriers to be studied to give the desired ratio of materials. A pH 6.8 phosphate buffer was added as needed to bring the volume up to 10 mL. After all material was added to the cuvette, the cuvette was sealed, sonicated for 2 hours on two separate occasions and then shaken for at least 72 hours at 37° C. The resulting material was filtered through a 0.45 micron filter and analyzed by UV-Visible spectroscopy to determine the AKBA content. Results are given in Table 1 and FIG. 2.

### Example 3

Evaluation of Solubility Enhancement Capability of the Combination of Polymer Carriers (Thermal Binders) and Surfactants for Solubilizing AKBA.

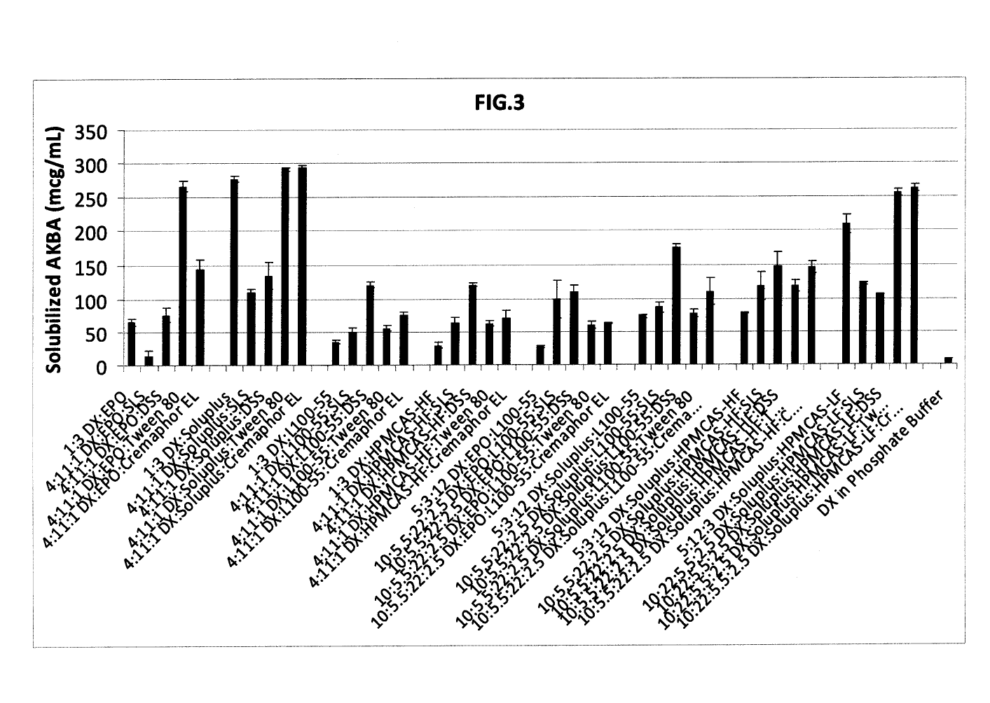

20 mg of AKBA was placed in a 10 mL cuvette, followed by the desired amount of polymer carriers and/or surfactants to be studied to give the desired ratio of materials. A pH 6.8 phosphate buffer was added as needed to bring the volume up to 10 mL. After all material was added to the cuvette, the cuvette was sealed, sonicated for 2 hours on two separate occasions and then shaken for at least 72 hours at 37° C. The resulting material was filtered through a 0.45 micron filter and analyzed by UV-Visible spectroscopy to determine the AKBA content. Results are given in Table 1 and FIG. 3.

TABLE 1
Solubility enhancements of acetyl-11-keto-β-boswellic acid (AKBA), given
in microgram per milliliter of pH 6.8 phosphate buffer. The material composition
is described by the ratio of AKBA to additives comprising the composition.

| | | | | Material - | |
|---|---|---|---|---|---|
| | | | | Combination of | |
| Material - | mcg/mL | Material - | mcg/mL | Surfactants and | mcg/mL |
| Surfactants | Buffer | Polymer Carriers | Buffer | Polymer Carriers | Buffer |
| AKBA | 0.01 | 1:3 | 44.29 | 1:3 | 64.67 |
| | | AKBA:EPO | | AKBA:EPO | |
| | | 1:1:2 | 63.23 | 4:11:1 | 13.87 |
| | | AKBA:EPO:HPMCAS-LF | | AKBA:EPO:SLS | |
| 10:1 | 15.04 | 1:1:2 | 53.16 | 4:11:1 | 75.99 |
| AKBA:SLS | | AKBA:EPO:HPMCAS-MF | | AKBA:EPO:DSS | |
| 4:1 | 42.40 | 1:1:2 | 47.62 | 4:11:1 | 266.72 |
| AKBA:SLS | | AKBA:EPO:HPMCAS-HF | | AKBA:EPO:Tween 80 | |
| 2:1 | 35.34 | 1:1:2 | 78.33 | 4:11:1 | 143.70 |
| AKBA:SLS | | AKBA:EPO:L100-55 | | AKBA:EPO:Cremaphor EL | |
| 10:1 | 25.24 | 5:3:12 | 70.67 | 1:3 | 276.42 |
| AKBA:DSS | | AKBA:EPO:HPMCAS-LF | | AKBA:Soluplus | |
| 4:1 | 70.83 | 5:3:12 | 48.19 | 4:11:1 | 109.61 |
| AKBA:DSS | | AKBA:EPO:HPMCAS-MF | | AKBA:Soluplus:SLS | |
| 2:1 | 115.35 | 5:3:12 | 50.82 | 4:11:1 | 134.48 |
| AKBA:DSS | | AKBA:EPO:HPMCAS-HF | | AKBA:Soluplus:DSS | |
| | | 5:3:12 | 91.60 | 4:11:1 | 291.07 |
| | | AKBA:EPO:L100-55 | | AKBA:Soluplus:Tween 80 | |
| 10:1 | 0.22 | | | 4:11:1 | 293.26 |
| AKBA:Gelucire 50/13 | | | | AKBA:Soluplus | |
| | | | | Cremaphor EL | |
| 4:1 | 0.48 | 1:3 | 27.27 | | |
| AKBA:Gelucire 50/13 | | AKBA:HPMCAS-LF | | | |
| 2:1 | 0.71 | 1:1:2 | 251.95 | 1:3 | 35.16 |
| AKBA:Gelucire 50/13 | | AKBA:HPMCAS- | | AKBA:L100-55 | |
| | | LF:Soluplus | | | |
| | | 5:3:12 | 291.38 | 4:11:1 | 50.44 |
| | | AKBA:HPMCAS- | | AKBA:L100-55:SLS | |
| | | LF:Soluplus | | | |
| 10:1 | 0.29 | 1:2:1 | 127.55 | 4:11:1 | 120.88 |
| AKBA:Gelucire 44/14 | | AKBA:HPMCAS- | | AKBA:L100-55:DSS | |
| | | LF:Soluplus | | | |
| 4:1 | 0.39 | 5:12:3 | 101.74 | 4:11:1 | 55.52 |
| AKBA:Gelucire 44/14 | | AKBA:HPMCAS- | | AKBA:L100-55:Tween 80 | |
| | | LF:Soluplus | | | |
| 2:1 | 0.54 | 1:3 | 28.90 | 4:11:1 | 74.96 |
| AKBA:Gelucire 44/14 | | AKBA:HPMCAS-MF | | AKBA:L100- | |
| | | | | 55:Cremaphor EL | |
| 10:1 | 8.92 | 1:1:2 | 241.03 | 1:3 | 29.66 |
| AKBA:Tween 80 | | AKBA:HPMCAS- | | AKBA:HPMCAS-HF | |
| | | MF:Soluplus | | | |
| 4:1 | 30.04 | 5:3:12 | 273.23 | 4:11:1 | 62.85 |
| AKBA:Tween 80 | | AKBA:HPMCAS- | | AKBA:HPMCAS- | |
| | | MF:Soluplus | | HF:SLS | |
| 2:1 | 57.02 | 1:2:1 | 153.44 | 4:11:1 | 120.75 |
| AKBA:Tween 80 | | AKBA:HPMCAS- | | AKBA:HPMCAS- | |
| | | MF:Soluplus | | HF:DSS | |
| | | 5:12:3 | 104.57 | 4:11:1 | 62.55 |
| | | AKBA:HPMCAS- | | AKBA:HPMCAS- | |
| | | MF:Soluplus | | HF:Tween 80 | |
| 10:1 | 14.08 | | | 4:11:1 | 71.19 |
| AKBA:Cremaphor EL | | | | AKBA:HPMCAS- | |
| | | | | HF:Cremaphor EL | |
| 4:1 | 41.29 | 1:3 | 36.91 | | |
| AKBA:Cremaphor EL | | AKBA:HPMCAS-HF | | | |
| 2:1 | 90.14 | 1:1:2 | 259.40 | 5:3:12 | 27.76 |
| AKBA:Cremaphor EL | | AKBA:HPMCAS- | | AKBA:EPO:L100-55 | |
| | | HF:Soluplus | | | |
| | | 5:3:12 | 299.40 | 10:5.5:22:2.5 | 99.20 |
| | | AKBA:HPMCAS- | | AKBA:EPO:L100- | |
| | | HF:Soluplus | | 55:SLS | |
| 10:1 | 7.41 | 1:2:1 | 159.40 | 10:5.5:22:2.5 | 110.54 |
| AKBA:Cremaphor RH | | AKBA:HPMCAS- | | AKBA:EPO:L100- | |
| | | HF:Soluplus | | 55:DSS | |
| 4:1 | 20.35 | 5:12:3 | 96.56 | 10:5.5:22:2.5 | 60.22 |
| AKBA:Cremaphor RH | | AKBA:HPMCAS- | | AKBA:EPO:L100- | |
| | | HF:Soluplus | | 55:Tween 80 | |
| 2:1 | 41.39 | 1:3 | 62.16 | 10:5.5:22:2.5 | 62.70 |
| AKBA:Cremaphor RH | | AKBA:L100-55 | | AKBA:EPO:L100- | |
| | | | | 55:Cremaphor EL | |
| 10:1 | 5.95 | 1:1:2 | 226.91 | 5:3:12 | 72.98 |
| AKBA:Solutol HS 15 | | AKBA:L100- | | AKBA:Soluplus:L100-55 | |
| | | 55:Soluplus | | | |
| 4:1 | 21.51 | 5:3:12 | 337.55 | 10:5.5:22:2.5 | 86.91 |
| AKBA:Solutol HS 15 | | AKBA:L100- | | AKBA:Soluplus:L100- | |
| | | 55:Soluplus | | 55:SLS | |
| 2:1 | 39.84 | 1:2:1 | 222.66 | 10:5.5:22:2.5 | 175.65 |
| AKBA:Solutol HS 15 | | AKBA:L100- | | AKBA:Soluplus:L100- | |
| | | 55:Soluplus | | 55:DSS | |
| | | 5:12:3 | 191.74 | 10:5.5:22:2.5 | 77.73 |
| | | AKBA:L100- | | AKBA:Soluplus:L100- | |
| | | 55:Soluplus | | 55:Tween 80 | |
| 10:1 | 0.86 | | | 10:5.5:22:2.5 | 110.63 |
| AKBA:Labrifil | | | | AKBA:Soluplus:L100- | |
| M 1944 CS | | | | 55:Cremaphor EL | |
| 4:1 | 0.47 | 1:3 | 395.14 | | |
| AKBA:Labrifil | | AKBA:Soluplus | | | |
| M 1944 CS | | | | | |
| 2:1 | 0.41 | | | 5:3:12 | 78.68 |
| AKBA:Labrifil | | | | AKBA:Soluplus:HPMC | |
| M 1944 CS | | | | AS-HF | |
| | | AKBA | 7.27 | 10:5.5:22:2.5 | 118.01 |
| | | | | AKBA:Soluplus:HPMC | |
| | | | | AS-HF:SLS | |
| | | | | 10:5.5:22:2.5 | 146.78 |
| | | | | AKBA:Soluplus:HPMC | |
| | | | | AS-HF:DSS | |
| | | | | 10:5.5:22:2.5 | 118.31 |
| | | | | AKBA:Soluplus:HPMC | |
| | | | | AS-HF:Tween 80 | |
| | | | | 10:5.5:22:2.5 | 145.45 |
| | | | | AKBA:Soluplus:HPMC | |
| | | | | AS-HF:Cremaphor EL | |
| | | | | 5:12:3 | 209.47 |
| | | | | AKBA:Soluplus:HPMC | |
| | | | | AS-LF | |
| | | | | 10:22:5.5:2.5 | 121.45 |
| | | | | AKBA:Soluplus:HPMC | |
| | | | | AS-LF:SLS | |
| | | | | 10:22:5.5:2.5 | 105.54 |
| | | | | AKBA:Soluplus:HPMC | |
| | | | | AS-LF:DSS | |
| | | | | 10:22:5.5:2.5 | 255.75 |
| | | | | AKBA:Soluplus:HPMC | |
| | | | | AS-LF:Tween 80 | |
| | | | | 10:22:5.5:2.5 | 262.91 |
| | | | | AKBA:Soluplus:HPMC | |
| | | | | AS-LF: Cremaphor EL | |
| | | | | AKBA in Phosphate | 8.46 |
| | | | | Buffer | |
| | | | | 10:1 | 112.05 |
| | | | | AKBA:Span 20 | |
| | | | | 4:1 | 258.23 |
| | | | | AKBA:Span 20 | |
| | | | | 2:1 | 282.53 |
| | | | | AKBA:Span 20 | |

### Example 4

Formulations of AKBA

Selected formulations of AKBA were produced through TKC and by roto-evaporation. For roto-evaporation, the materials to be combined into the formulation were dissolved in excess alcohol solvent (5 gm batches in 150 ml of alcohol solvent) to dissolve all the material, placed into a flask and attached to a roto-evaporation unit (Buchi Rotavapor® R-210/R-215). The alcohol solvent was removed under reduced pressure at a temperature of 60-80° C. to produce the formulation. The formulations produced utilizing roto-evaporation are listed in Table 2.

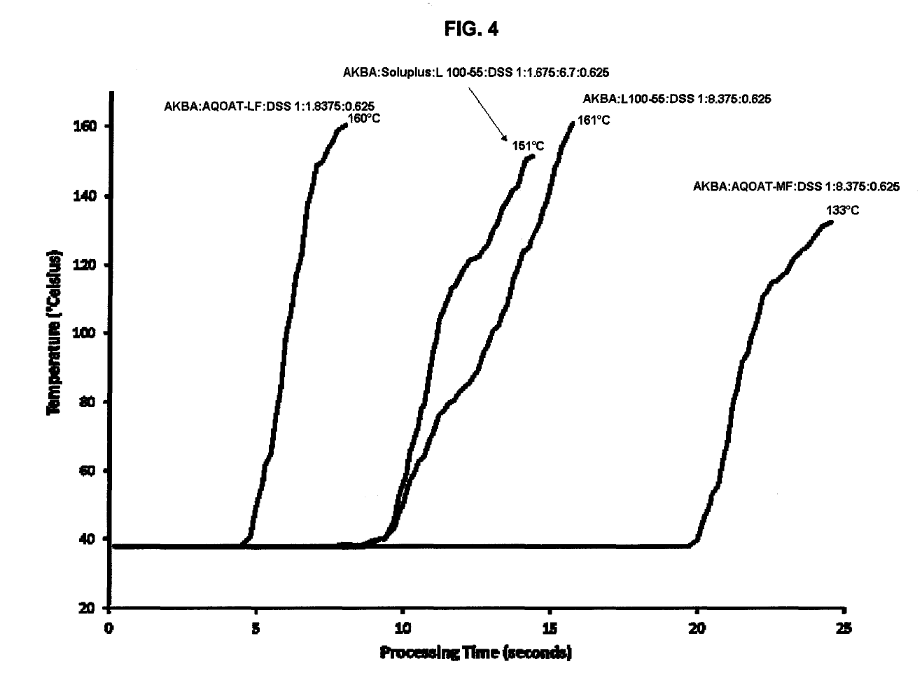

For thermokinetic compounding, the materials (60 gm batches) to be combined into the formulation were placed into a TKC chamber and processed at a set speed (RPMs) to a set ejection temperature, and then cooled. The conditions used and the formulations produced utilizing TKC are listed in Table 3. Process data from the production of the formulations using TKC is shown in FIG. 4.

TABLE 2
Formations of AKBA produced using roto-evaporation (5
gm batches dissolved in 150 mL of alcohol solvent).

| Batch ID | Formulation Contents | Formulation Ratio |
|---|---|---|
| 001 | AKBA:AQOAT-LF:DSS | 1:1.8375:0.625 |
| 002 | AKBA:AQOAT-MF:DSS | 1:8.375:0.625 |
| 003 | AKBA:L100-55:DSS | 1:8.375:0.625 |
| 004 | AKBA:E PO:Tween 80 | 1:8.8:0.2 |
| 005 | AKBA:Soluplus:L 100-55:DSS | 1:1.675:6.7:0.625 |
| 006 | AKBA:E PO:L100-55:DSS | 1:1.675:7.0:0.625 |

TABLE 3
Formulations of AKBA produced using thermokinetic
compounding (60 gm batches).

| | | | | Temper- | |
|---|---|---|---|---|---|
| | | | | ature (° C.) | |
| Batch | Formulation | Formulation | | | Ejec- |
| ID | Contents | Ratio | RPMs | Set | tion |
| 007 | AKBA:AQOAT- | 1:1.8375:0.625 | 3000 | 132 | 160 |
| | LF:DSS | | | | |
| 008 | AKBA:AQOAT- | 1:8.375:0.625 | 2600 | 132 | 166 |
| | MF:DSS | | | | |
| 009 | AKBA:Soluplus:L | 1:1.675:6.7:0.625 | 3000 | 138 | 151 |
| | 100-55:DSS | | | | |
| 010 | AKBA:L100- | 1:8.375:0.625 | 3000 | 138 | 161 |
| | 55:DSS | | | | |

### Example 5

Characterization Studies of the Formulations of AKBA.

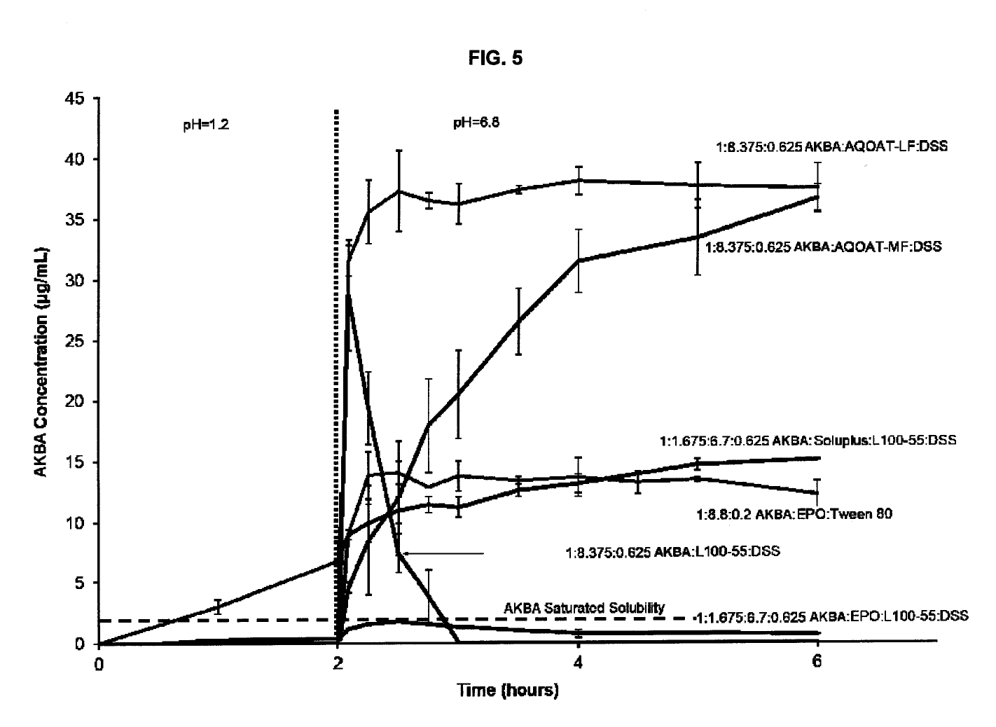

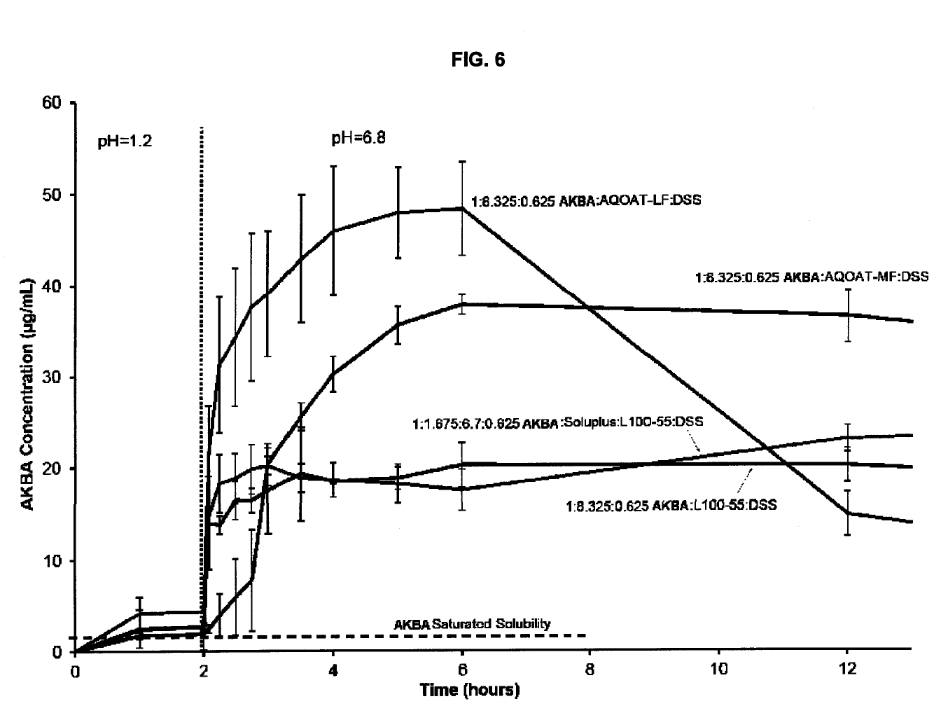

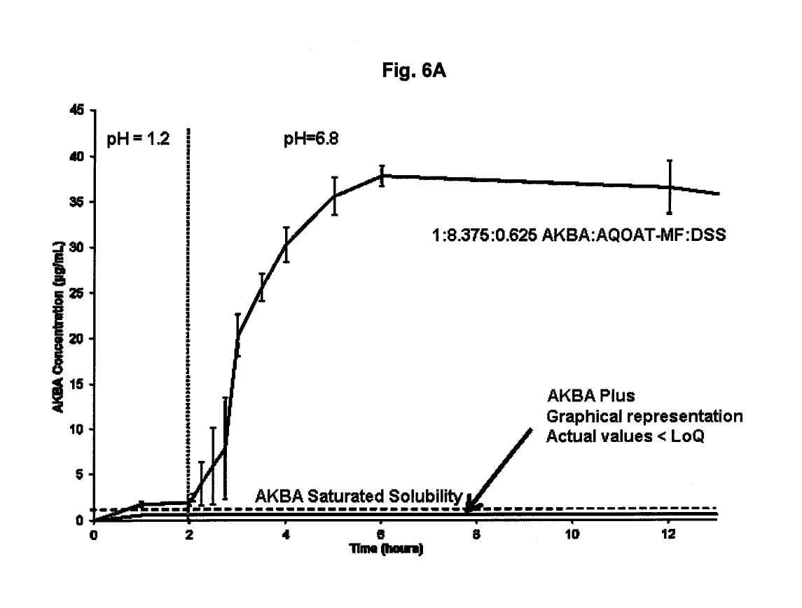

Dissolution tests were performed with a dissolution apparatus (VK 7000 Dissolution Tester with a VK 8000 Autosampler). The testing was carried out according to a published method (USP XXIX method A enteric dissolution test); non-sink conditions were maintained throughout the testing using 1 L of final neutral media. The solution of 0.1N HCl (pH of 1.2) with a formulation present was stirred at 50 rpm for 2 hours and then the solution was converted to 0.2M Phosphate Buffer (pH of 6.8) and stirring continued up to 12 hours. At times of 60, 120, 125, 135, 150, 165, 180, 210, 240, 300, 360, 480, and 720 minutes, three×5 mL samples were removed and analyzed for soluble AKBA. The results of the dissolution studies are the displayed in FIG. 5 for the formulations produced by roto-evaporation and in FIG. 6 for formulations produced by TKC. Direct comparison of dissolution results of a formulation by TKC with a marketed AKBA product (AKBA Plus) are displayed in FIG. 6A. As can be ascertained from FIG. 6, our reference standard AKBA has a peak solubility of about 2 μg/ml, whereas the peak solubility of the formulations produced by TKC as disclosed herein range from about 20 μg/ml to about 48 μg/ml in a pH 6.8 buffer.

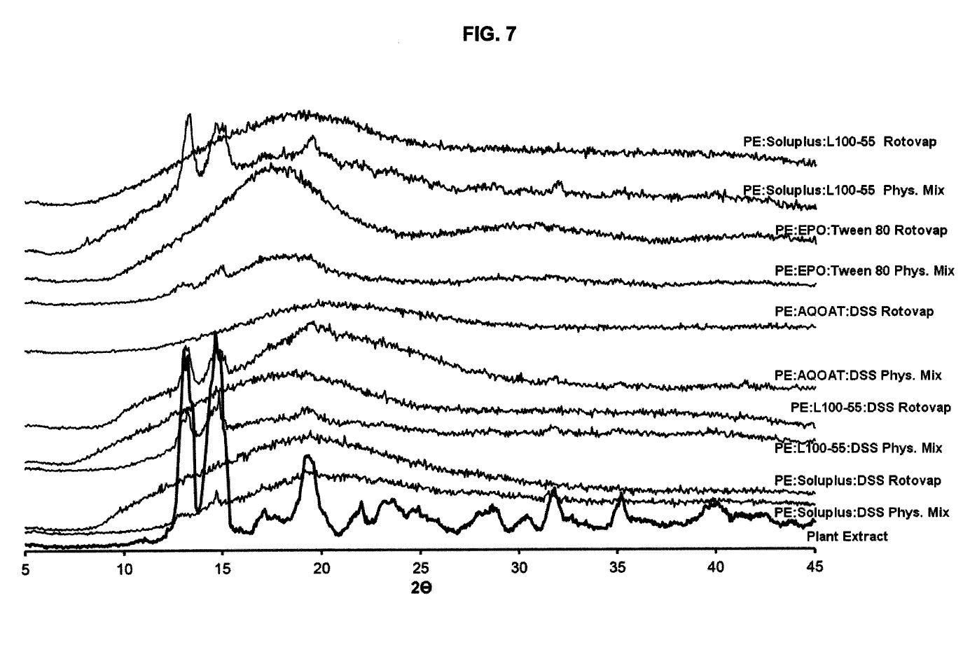

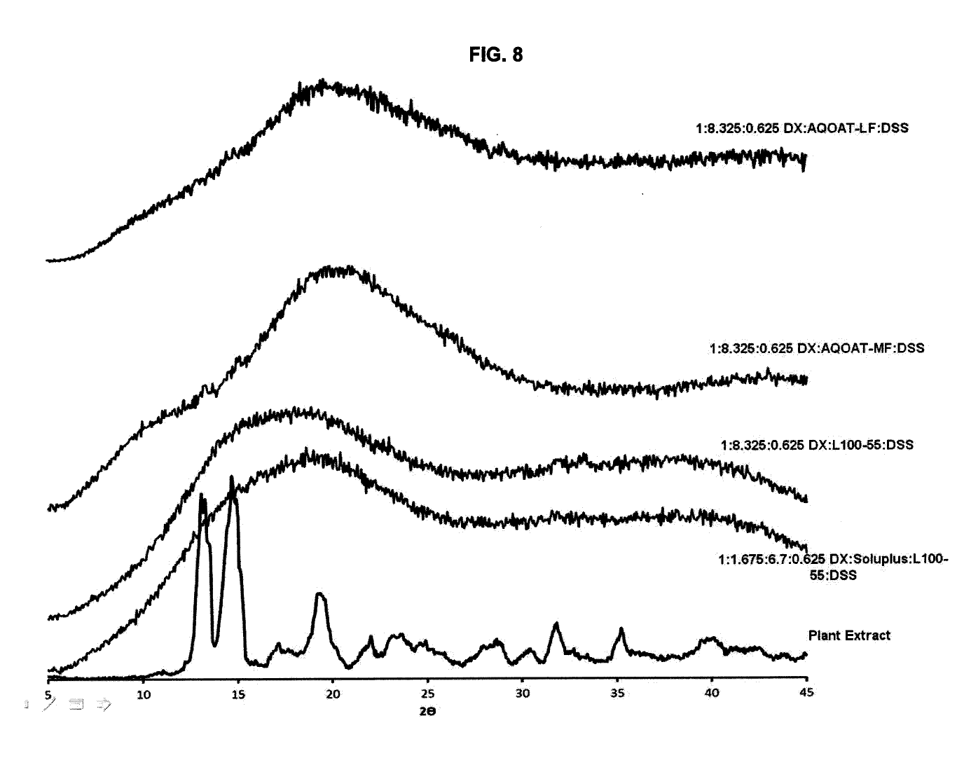

X-Ray diffraction (XRD) measurements of the formulations were carried out on an XRD system (Philips Model 1710 X-Ray Diffractometer). The parameters used were a 2θ scan range of 5° to 45°, with a step size of 0.05° and a dwell Time of 5 seconds. The measured XRD patterns are shown in FIG. 7 for formulations produced by roto-evaporation and are shown in FIG. 8 for formulations produced with TKC.

High performance liquid chromatography was used to determine AKBA recovery in TKC processed samples. Milled dispersions were accurately weighed to 150.0±3 mg and placed in 100-mL volumetric flasks. Approximately 90 mL of a 8:2:0.4 acetonitrile:water:glacial acetic acid diluent mixture was added to the flasks which were then subjected to 2 min of sonication. Once brought to volume and mixed, samples were filtered through 25 mm 0.2 μm PVDF filters (Whatman, Piscataway, N.J.) and transferred to 1 mL vials (VWR International, West Chester, Pa.) for HPLC analysis. AKBA assay values were adjusted for the recorded sample weight and compared to a known standard containing 15.0±0.10 mg in 100 mL of the diluent described above.

High performance liquid chromatography (HPLC) was used to analyze AKBA content in the recovery assay samples. A Waters 717 autosampler (Waters Corporation, Milford, Mass.) was utilized to inject 20 μL samples onto a Phenomenex® luna 5 μm C18(2), 150 mm×4.6 mm column (Phenomenex®, Torrance, Calif.). The aqueous mobile phase consisted of 8:2:0.4 acetonitrile:water: glacial acetic acid. The total mobile phase flow rate throughout the gradient method was 2 mL min-1. A Waters 2996 photodiode array detector, extracting at 260 nm, quantified the amount of AKBA in each sample. The retention time of AKBA was approximately 7 min. All analyses maintained linearity (R2≧0.999) in the range tested and a relative standard deviation of less than 2.0%. Empower version 5.0 was utilized to process all chromatography data. Analysis data of the recoverable AKBA content from the formulations produced using TKC is given in Table 4.

Analysis data of the recoverable AKBA content from the formulations produced using TKC is given in Table 4.

TABLE 4
Recover of AKBA from the formulations produced
using Thermo-Kinetic Compounding.

| Batch | | Formulation | AKBA Recovery |
|---|---|---|---|
| ID | Formulation Contents | Ratio | Assay (%) |
| 007 | AKBA:AQOAT-LF:DSS | 1:1.8375:0.625 | 100.71 ± 1.64 |
| 008 | AKBA:AQOAT-MF:DSS | 1:8.375:0.625 | 99.59 ± 1.21 |
| 009 | AKBA:Soluplus:L | 1:1.675:6.7:0.625 | 99.91 ± 0.67 |
| | 100-55:DSS | | |
| 010 | AKBA:L100-55:DSS | 1:8.375:0.625 | 99.18 ± 0.32 |

### Example 6

Bioavailability Study of the Formulations of AKBA.

A study was performed to evaluate the pharmacokinetic parameters of AKBA compositions. For the study, adult male Sprague-Dawley rats (275-300 grams, 63-67 days) were caged individually and maintained on a normal rodent chow diet with free access to water. Rats were randomly divided into groups of 4 rats with each group receiving one formulation. Prior to dosing, each formulation was reconstituted in deionised water. The formulations were administered as a dose equivalent to 50 mg/kg AKBA. Each formulation was delivered by 1 mL oral gavage via a pharyngeal tube such that each group of 4 rats received a different formulation. Each formulation was administered to dose AKBA at a concentration of 15 mg/l mL to prevent spontaneous release through the pyloric sphincter (volumetric dose below 4 mL/kg body weight).

The administered formulations were:

1 Control Drug (Commercial AKBA product—“AKBA Plus”)

2. AKBA:AQOAT-LF:DSS (1:1.8375:0.625) (Batch 007)

3 AKBA:AQOAT-MF:DSS (1:8.375:0.625) (Batch 008)

4. AKBA:Soluplus:L 100-55:DSS (1:1.675:6.7:0.625) (Batch 009)

5 AKBA:L100-55: DSS (1:8.375:0.625) (Batch 10)

6 Raw AKBA

Blood Samples:

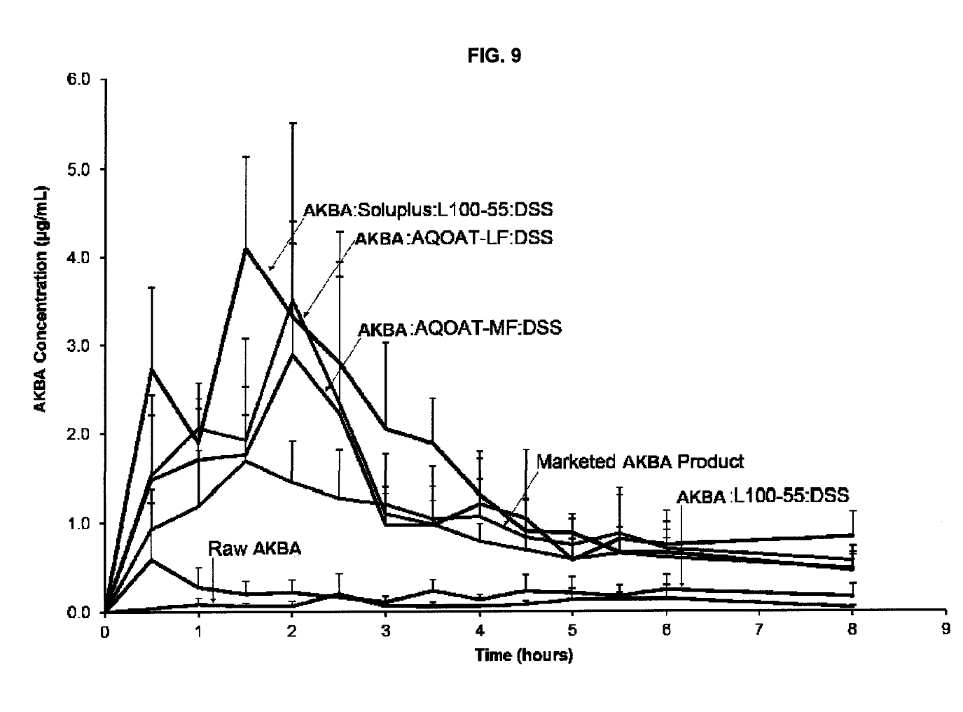

The catheters of the rats were flushed daily with 300 μL of 50 U/mL heparinized normal saline. Blood samples of approximately 300 μL were collected from the jugular vein catheter at 0, 0.5, 1, 1.5, 2, 2.5, 3, 3.5, 4, 4.5, 5, 5.5, 6 and 8 hours after dosing using a 1 mL syringe and attached 25 gram needle. Blood was slowly extracted into the needle through the catheter. The blood samples were placed into preheparinized 1.5 mL microcentrifuge tubes. Withdrawn blood was replaced with equal volumes of heparinized saline by injecting through the jugular vein catheter. The bioavailability of the API was then determined by monitoring the API levels in the blood over time. The results of the study are presented in Table 5 and displayed in FIG. 9.

The results of the PK study demonstrate that, with the exception of one formulation, all solubility enhanced AKBA compositions produced substantial improvements in oral exposure over AKBA itself. Thus demonstrating that the oral bioavailability of AKBA could be markedly improved via solubility enhanced formulations.

TABLE 5
Results of the in vivo testing to assess bioavailability
of AKBA of various formulations.

| | | | Average | |
|---|---|---|---|---|
| Adminis- | Average | Average | of AUC | Average |
| tered For- | of Cₘₐₓ | of Tₘₐₓ | (μg-hr/ | of t_{1/2} |
| mulation | (μg/mL) | (hours) | mL) | (hours) |
| Marketed | 1.46 ± 0.61 | 1.81 ± 0.61 | 7.50 ± 3.23 | 2.72 ± 0.71 |
| AKBA | | | | |
| Batch 007 | 4.17* ± 1.56 | 1.88 ± 0.63 | 9.32 ± 2.08 | 5.68* ± 1.92 |
| Batch 008 | 3.73* ± 1.43 | 2.13 ± 0.25 | 9.58 ± 3.47 | 4.17 ± 1.64 |
| Batch 009 | 4.10* ± 1.03 | 1.63 ± 0.25 | 12.55 ± 3.00 | 3.12 ± 0.67 |
| Batch 010 | 0.90 ± 0.77 | 0.61 ± 0.93 | 1.45* ± 0.85 | 1.26 ± 1.28 |
| Raw | 0.35* ± 0.12 | 4.75* ± 1.55 | 0.71* ± 0.24 | 2.30 ± 1.43 |
| AKBA | | | | |

*Statistically significant

### Example 7

Further Bioavailability Studies of the Compositions of the Present Disclosure.

A further study will be performed to evaluate the pharmacokinetic parameters of AKBA compositions of the present disclosure. For the study, adult male Sprague-Dawley rats will be caged individually and maintained on a normal rodent chow diet with free access to water. Rats will be randomly divided into 8 groups of 6 rats based on each group receiving one formulation. Prior to dosing, each formulation will be reconstituted in deionised water. The formulations will be administered as a dose equivalent to 50 mg/kg AKBA. Each formulation will be delivered by 1 mL oral gavage via a pharyngeal tube such that each of 8 groups receives a different formulation. Each formulation will be administered to dose AKBA at a concentration of 15 mg/l mL to prevent spontaneous release through the pyloric sphincter (volumetric dose below 4 mL/kg body weight).

The administered formulations will be:

1-5.25% w/w Drug:Polymer(s):Surfactant

6. AKBA Plus (marketed product)

7. Control Drug (bulk material)

8. Oral placebo formulation (drug free)

Formulations 1-5 will contain 25% AKBA by total weight. Formulations 1-5 may be comprised of any polymer or combination of polymers listed below. Formulations 1-5 may or may not be comprised of a surfactant from those listed below. The formulations for testing in the current example may be processed through thermokinetic compounding, thermokinetic compounding followed by another processing method known in the art, or by another processing method known in the art to produce the material for testing.

Polymers that will be incorporated into the above formulations include the following: Soluplus™ (Manufacturer: BASF), Eudragit™ L100-55 (Evonik), Eudragit™ E PO (Evonik), Kollidon™ VA 64 (BASF), Kollidon™ (BASF), AQOAT™-HF and AQOAT™-LF (Shin-Etsu Chemical Co.).

Surfactants that may be incorporated into the above formulations include: Sodium dodecyl sulfate, Dioctyl docusate sodium, Tween 80, Span 20, Cremaphor™ EL, or Vitamin E TPGS.

Blood Samples:

The catheters of the rats will be flushed daily with 300 μL of 50 U/mL heparinized normal saline. Blood samples of approximately 300 μL will be collected from the jugular vein catheter at 0, 0.5, 1, 1.5, 2, 2.5, 3, 3.5, 4, 4.5, 5, 5.5, 6, 8, 12, 24, and 36 hours after dosing using a 1 mL syringe and attached 25 gram needle. Blood will be slowly extracted into the needle through the catheter. The blood samples will be placed into preheparinized 1.5 mL microcentrifuge tubes. Withdrawn blood will be replaced with equal volumes of heparinized saline by injecting through the jugular vein catheter. The bioavailability of the API will then be determined by monitoring the API levels in the blood over time.

### Example 8

Bioavailability Studies of Pharmaceutical Compositions of the Present Disclosure with Adjuvant Enzyme Inhibitors.

The effect of co-administering various enzyme inhibitors and AKBA will be evaluated for AKBA solubility enhancement as described herein. The study will compare the effects of both a broad-spectrum inhibitor (piperine) and more narrow-acting inhibitors on intestinal metabolism of AKBA. The goal of the study is to select AKBA formulations that optimize systemic bioavailability of AKBA.

A further study will be performed to evaluate the pharmacokinetic parameters of pharmaceutical compositions of the present disclosure comprising both AKBA and various enzyme inhibitors. For the study, adult male Sprague-Dawley rats will be caged individually and maintained on a normal rodent chow diet with free access to water. Rats will be randomly divided into groups of 6 rats, with each group receiving a different pharmaceutical formulation. Prior to dosing, each formulation will be reconstituted in deionised water. The formulations will be administered as a dose equivalent to 50 mg/kg AKBA, along with a corresponding dose of an enzyme inhibitor. Each formulation will be delivered by 1 mL oral gavage via a pharyngeal tube. Each formulation will be administered to dose AKBA at a concentration of 15 mg/mL to prevent spontaneous release through the pyloric sphincter (volumetric dose below 4 mL/kg body weight).

The different administered formulations will be:
1. AKBA Plus (marketed product)
2. AKBA:AQOAT-MF:DSS (1:8.375:0.625) (Batch 008) with piperine (1 mg/kg)
3. AKBA:AQOAT-MF:DSS (1:8.375:0.625) (Batch 008) with concentrated grapefruit juice (10 mL/kg administered 2 hrs before AKBA formulation)
4. AKBA:AQOAT-MF:DSS (1:8.375:0.625) (Batch 008) with ritonavir oral solution (10 mg/kg)
5. AKBA:AQOAT-MF:DSS (1:8.375:0.625) (Batch 008) with α-naphthoflavone (10 mg/kg)
6. AKBA:AQOAT-MF:DSS (1:8.375:0.625) (Batch 008) with quinidine (10 mg/kg)

Blood Samples:

The catheters of the rats will be flushed daily with 300 μL of 50 U/mL heparinized normal saline. Blood samples of approximately 300 μL will be collected from the jugular vein catheter at 0, 0.5, 1, 1.5, 2, 2.5, 3, 3.5, 4, 4.5, 5, 5.5, 6, 8, 12, 24, and 36 hours after dosing using a 1 mL syringe and attached 25 gram needle. Blood will be slowly extracted into the needle through the catheter. The blood samples will be placed into pre-heparinized 1.5 mL microcentrifuge tubes. Withdrawn blood will be replaced with equal volumes of heparinized saline by injecting through the jugular vein catheter. The bioavailability of the AKBA will then be determined by monitoring the API levels in the blood over time.

The first formulation, AKBA Plus, will serve as the control for this experiment, i.e., non-solubility enhanced formulation of AKBA with a broad spectrum intestinal enzyme inhibitor. The AKBA:AQOAT-MF:DSS (1:8.375:0.625) formulation was selected for the non-control arms of this study as it was determined to be the most viable of the solubility enhanced formulations of AKBA produced to date. In the second arm of this study the AKBA formulation will be co-administered with piperine in an equivalent amount as is contained in AKBA Plus arm (1 mg/kg). This arm is intended to prove the hypothesis that the combination of a solubility enhanced formulation of AKBA as disclosed herein and an inhibitor of intestinal enzymes will improve systemic levels of AKBA. In the third arm of the study, the solubility enhanced formulation of AKBA will be dosed two hours after administration of concentrated grapefruit juice. Grapefruit juice is a known inhibitor of intestinal cytochrome P450 enzymes, thus improvements in systemic exposure of AKBA resulting from administration of grapefruit juice will indicate that intestinal cytochrome P450s are the key class of enzymes responsible for the metabolism of AKBA. In the fourth arm of the study, the solubility enhanced AKBA formulation will be co-administered with the known CYP3A inhibitor ritonavir. Improvements in systemic exposure resulting from ritonavir will indicate that the CYP3A family of enzymes (a sub-class of CYP450) is involved in intestinal metabolism of AKBA. In the fifth arm of the study the solubility enhanced AKBA formulation will be co-administered with the known CYP1A1/1A2 inhibitor α-naphthoflavone. Improvements in systemic exposure resulting from α-naphthoflavone will indicate that the CYP1A family of enzymes (a sub-class of CYP450) is involved in intestinal metabolism of AKBA. In the sixth arm of the study, the solubility enhanced AKBA formulation will be co-administered with the known CYP2D6 inhibitor quinidine. Improvements in systemic exposure resulting from quinidine will indicate that the CYP2D family of enzymes (a sub-class of CYP450) is involved in intestinal metabolism of AKBA.

Expected Results
Arm 1: Similar results as in Example 6 of AKBA Plus.
Arm 2: Increases in exposure several fold greater than that achieved by AKBA Plus and the AKBA:AQOAT-MF:DSS (1:8.375:0.625) formulation administered without an enzyme inhibitor (Example 6).
Arm 3: Increases in exposure several fold greater than that achieved by AKBA Plus and the AKBA:AQOAT-MF:DSS (1:8.375:0.625) formulation administered without an enzyme inhibitor (Example 6).
Arm 4: Increases in exposure several fold greater than that achieved by AKBA Plus and the AKBA:AQOAT-MF:DSS (1:8.375:0.625) formulation administered without an enzyme inhibitor (Example 6).
Arm 5: Systemic exposures similar to the AKBA:AQOAT-MF:DSS (1:8.375:0.625) formulation administered without an enzyme inhibitor (Example 6).
Arm 6: Systemic exposures similar to the AKBA:AQOAT-MF:DSS (1:8.375:0.625) formulation administered without an enzyme inhibitor (Example 6).

The results of this study are expected to demonstrate that solubility enhanced formulations of AKBA as disclosed here, when co-administered with a CYP450 inhibitor, specifically an inhibitor of the CYP3A family, increase the bioavailability of AKBA. The systemic exposure from a composition containing a solubility enhanced formulation of AKBA and a CYP450 inhibitor, more specifically a CYP3A inhibitor, is expected to be several-fold greater than the solubility enhanced formulation alone, AKBA Plus, or a summation of exposures of the two.

1. A pharmaceutical composition comprising an active pharmaceutical ingredient selected from the group consisting of acetyl-11-keto-β-boswellic acid, diindolylmethane, and curcumin, and one or more pharmaceutically acceptable excipients, wherein a peak solubility of the active pharmaceutical ingredient in the composition is greater than 6 μg/mL in an aqueous buffer with a pH range of 4 to 8.

2. A pharmaceutical composition comprising an active pharmaceutical ingredient selected from the group consisting of acetyl-11-keto-β-boswellic acid, diindolylmethane, and curcumin, and one or more pharmaceutically acceptable excipients, wherein peak solubility of the active pharmaceutical ingredient in the composition and peak solubility of the reference standard active pharmaceutical ingredient in an aqueous buffer with a pH range of 4 to 8 have a ratio of greater than 3:1.

3. The composition of claim 2, wherein the ratio is greater than 10:1, greater than 20:1, or greater than 30:1.

4.-5. (canceled)

6. A pharmaceutical composition comprising an active pharmaceutical ingredient selected from the group consisting of acetyl-11-keto-β-boswellic acid, diindolylmethane, and curcumin, and one or more pharmaceutically acceptable excipients, wherein Cmax of the active pharmaceutical ingredient in the composition and Cmax of the reference standard active pharmaceutical ingredient have a ratio that is greater than 6:1.

7. The composition of claim 6, wherein the composition is delivered orally.

8. The composition of claim 6, wherein the ratio is greater than 10, greater than 15, or greater than 20.

9.-10. (canceled)

11. A pharmaceutical composition comprising an active pharmaceutical ingredient selected from the group consisting of acetyl-11-keto-β-boswellic acid, diindolylmethane, and curcumin, and one or more pharmaceutically acceptable excipients, wherein the composition is a homogenous, heterogenous, or heterogeneously homogenous composition in which the glass transition temperature is higher than the glass transition temperature of an identical combination of the active pharmaceutical ingredient and pharmaceutically acceptable excipients processed thermally.

12. A pharmaceutical composition comprising an active pharmaceutical ingredient selected from the group consisting of acetyl-11-keto-β-boswellic acid, diindolylmethane, and curcumin, and one or more pharmaceutically acceptable excipients, wherein the composition is a homogenous, heterogenous, or heterogeneously homogenous composition which has a single glass transition temperature, wherein an identical combination of the active pharmaceutical ingredient and pharmaceutically acceptable excipients processed thermally has two or more glass transition temperatures.

13. The composition of claim 11, wherein the identical combination is processed by hot melt extrusion.

14. The composition of claim 11, wherein the identical combination is processed with a plasticizer.

15. The composition of claim 11, wherein the composition has a single glass transition temperature that is at least 10% higher than the lowest glass transition temperature of the identical combination or at least 20% higher than the lowest glass transition temperature of the identical combination.

16. The composition of claim 12, wherein the pharmaceutical composition has a single glass transition temperature that is at least 10% higher than the lowest glass transition temperature of the identical combination or at least 20% higher than the lowest glass transition temperature of the identical combination.

17. A pharmaceutical composition comprising an active pharmaceutical ingredient selected from the group consisting of acetyl-11-keto-β-boswellic acid, diindolylmethane, and curcumin, and one or more pharmaceutically acceptable excipients, wherein the active pharmaceutical ingredient is thermally labile, wherein the composition is a homogenous, heterogenous, or heterogeneously homogenous composition that has less than 1.0% degradation products of the active pharmaceutical ingredient.

18. The composition of claim 1, wherein the pharmaceutically acceptable excipient is a surfactant.

19. The composition of claim 1, wherein the pharmaceutically acceptable excipient is a polymer carrier.

20. The composition of claim 1, wherein the pharmaceutically acceptable excipients comprises one or more surfactants and one or more polymer carriers.

21. The composition of claim 1, wherein the pharmaceutically acceptable excipient is selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS and sorbitan laurate.

22. The composition of claim 1, wherein the pharmaceutically acceptable excipient is selected from the group consisting of poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer.

23. The composition of claim 1, wherein the pharmaceutically acceptable excipient is selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS and sorbitan laurate and the polymer carrier is selected from a group consisting of poly(vinylpyrrolidone), ethylacrylate-methylmethacrylate copolymer, poly(methacrylate ethylacrylate) (1:1) copolymer, hydroxypropylmethylcellulose acetate succinate, poly(butyl methacylate-co-(2-dimethylaminoethyl) methacrylate-co-methyl methacrylate) 1:2:1 and polyvinyl caprolactam-polyvinyl acetate-polyethylene glycol graft copolymer.

24. The composition of claim 1, wherein the composition further comprises an adjuvant.

25. The composition of claim 24, wherein the adjuvant is an enzyme inhibitor.

26. The composition of claim 25, wherein the enzyme inhibitor inhibits a cytochrome P-450 enzyme.

27. The composition of claim 26, wherein the cytochrome P-450 enzyme is CYP3A4.

28. The composition of claim 24, wherein the adjuvant is selected from the group consisting of piperine, grapefruit extract, ritonavir, or ketoconazole.

29. The composition of claim 1, wherein the active pharmaceutical ingredient is acetyl-11-keto-β-boswellic acid.

30. The composition of claim 1, wherein the active pharmaceutical ingredient is diindolylmethane.

31. The composition of claim 1, wherein the active pharmaceutical ingredient is curcumin.

32. The composition of claim 1, wherein the pharmaceutical composition is processed by thermokinetic compounding.

33. The composition of claim 1, wherein the pharmaceutical composition is processed by thermokinetic compounding.

34. The composition of claim 2, wherein the pharmaceutical composition is processed by thermokinetic compounding.

35. The composition of claim 11, wherein the pharmaceutical composition is processed by thermokinetic compounding.

36. The composition of claim 17, wherein the pharmaceutical composition is processed by thermokinetic compounding.

37. The composition of claim 1, wherein the pharmaceutically acceptable excipient is selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, sorbitan laurate, poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer.

38. The composition of claim 2, wherein the pharmaceutically acceptable excipient is selected from the group consisting of sodium dodecyl sulfate, dioctyl sodium sulphosuccinate, polyoxyethylene (20) sorbitan monooleate, glycerol polyethylene glycol oxystearate-fatty acid glycerol polyglycol esters-polyethylene glycols-glycerol ethoxylate, glycerol-polyethylene glycol ricinoleate-fatty acid esters of polyethyleneglycol-polyethylene glycols-ethoxylated glycerol, vitamin E TPGS, sorbitan laurate, poly(vinyl acetate)-co-poly(vinylpyrrolidone) copolymer, ethylcellulose, hydroxypropylcellulose, cellulose acetate butyrate, poly(vinylpyrrolidone), poly(ethylene glycol), poly(ethylene oxide), poly(vinyl alcohol), hydroxypropyl methylcellulose, ethylcellulose, hydroxyethylcellulose, sodium carboxymethyl-cellulose, dimethylaminoethyl methacrylate-methacrylic acid ester copolymer, ethylacrylate-methylmethacrylate copolymer, cellulose acetate phthalate, cellulose acetate trimelletate, poly(vinyl acetate) phthalate, hydroxypropylmethylcellulose phthalate, poly(methacrylate ethylacrylate) (1:1) copolymer, poly(methacrylate methylmethacrylate) (1:1) copolymer, poly(methacrylate methylmethacrylate) (1:2) copolymer, hydroxypropylmethylcellulose acetate succinate and polyvinyl caprolactam-polyvinyl acetate-polyethylene glycol graft copolymer.
